(19) United States
(12) Patent Application Publication
(10) Pub. No.: US 2026/0262609 A1
(43) Pub. Date: Sep. 10, 2026

(54) FUTURE ANIMAL HEALTH AND PERFORMANCE OPTIMIZATIZATION USING MULTIGENERATIONAL MICROBIOME ANALYTICS

(71) Applicant: CAN TECHNOLOGIES, INC., Wayzata, MN (US)

(72) Inventors: Anirikh Chakrabarti, Mechelen (BE); Ehsan Khafipour, Tampa, FL (US); Briana Kristen Kozlowicz, Hopkins, MN (US); Syed Ali Faraz Naqvi, Calgary (CA)

(73) Assignee: CAN TECHNOLOGIES, INC., Wayzata, MN (US)

(21) Appl. No.: 19/165,231

(22) PCT Filed: Mar. 14, 2024
(86) PCT No.: PCT/US2024/019849

§ 371 Date: Sep. 15, 2025

Related U.S. Application Data
(60) Provisional application No. 63/490,920, filed on Mar. 17, 2023.

(51) Int. Cl. A01K 29/00; A61D 99/00

(52) U.S. Cl. CPC A01K 29/004; A61D 99/00

(57) ABSTRACT

The present disclosure provides systems and methods for optimizing future health or future performance in an animal. The methods can include obtaining a sample dataset indicative of an entire microbial community within a gastrointestinal tract, mammary gland, reproductive system, or respiratory system. The methods can include defining a query for future health or future performance of the animal. The methods can include selecting a model from a model repository based on the sample dataset and the query. The method can include executing the selected model using the sample dataset and the query to make predictions, generate recommendations, or generate interventions about future health or future performance of the animal. The methods can include reporting the predictions, recommendations, or interventions about future health or future performance, and implementing one or more interventions by based on the predictions, recommendations, or interventions. Other aspects are also provided herein.

### CROSS-REFERENCE TO RELATED APPLICATIONS

This application claims the benefit of U.S. Provisional Patent Application No. 63/490,920, filed on 17 Mar. 2023, which is hereby incorporated by reference in its entirety.

### FIELD

The present disclosure relates to systems and methods for optimizing future animal health and performance. In specific, the present disclosure relates to systems and methods for optimizing future animal health and performance using metagenomics data and various metadata to make one or more predictions, recommendations, or interventions about future health measures or future performance measures of an animal or group of animals.

### BACKGROUND

The microbiome of pigs undergoes longitudinal and dynamic shifts during the lifespan of the pig. In some cases, the dynamic shifts provide beneficial outcomes to the pigs, while in other cases the dynamic shifts can lead to illness and poor performance measures. Beginning at birth and maturing into the sow life stage, the microbiome of pigs is continually shifting as influenced by the transitions and exposures experienced by the pig through its lifetime, including transitions from piglet to gilt to sow, during weaning, during farrowing, as impacted by environmental exposure or pathogens, a change in diet and nutrition management, use of antimicrobials, and so on. Large dynamic shifts in the pig microbiome can have significant negative impacts on the health and performance of the animals and can adversely affect a number of health and performance measures. Such health and performance measures can include general health of the animal, body weight, average daily weight gain, feed intake, feed conversion ratio, and many more. Without insight into the composition of the pig's microbiome throughout its lifespan, it can be difficult or impossible to tailor multiple aspects of the pig's rearing to address the effects of the dynamic shifts.

Past efforts for trying to address the problem of dynamic microbiome shifts include using a subset of biomarkers from retrospective metagenomics data obtained on the population level to targeting specific outcomes. Such methods shied away from using large sets of microbiome data on the individual animal level and thus did not utilize the entirety of the microbiome to leverage the impact dynamic shifts within the entire microbiome had on future health and performance of existing individual animals or a subset of a group of animals. Further, such methods did not use multigenerational data to predict the future health and performance of an individual animal or subset of a group of animals. Thus, a need exists to provide a system and methods for using large-scale metagenomics data for an existing animal(s) to predict that animal's(s') future health and performance, and that further can be utilized for individual selection of animals or group-level selection of a subset of animals to apply custom interventions so as to optimize the future health and future performance of the animal or subset of animals.

### SUMMARY

The present disclosure provides a method for optimizing future health or future performance in an animal. The method can include obtaining a sample dataset indicative of an entire microbial community within a gastrointestinal tract, mammary gland, reproductive system, or respiratory system of the animal. The method further can include defining a query including one or more target queries for future health or future performance of the animal. The method further can include selecting a model from a model repository based on the sample dataset and the query including the one or more targets. The method further can include executing the selected model using the sample dataset and the query including the one or more targets to make predictions, generate recommendations, or generate interventions about future health or future performance of the animal. The method further can include reporting the predictions, recommendations, or interventions about future health or future performance, or combinations thereof. The method further can include implementing one or more interventions as one or more adjustments to the animal's nutrition, management system, health care, or rearing environment based on the predictions, recommendations, or interventions.

The present disclosure provides a multigenerational microbiome system for optimizing future health or future performance in one or more animals. The multigenerational microbiome system can include: a metagenomics component configured to receive metagenomics data obtained from a microbiome sample of the one or more animals; a metadata acquisition component configured to receive metadata about the one or more animals; a query acquisition component configured to receive one or more target queries for future health or future performance of the one or more animals; and a processing component. The processing component of the multigenerational microbiome system can include a model selection engine, where the model selection engine is configured to select a model or set of models from a model repository based on the metagenomics data, the metadata, and the target query; a prediction generation engine, where the prediction generation engine is configured to execute the selected model or set of models to generate one or more predictions about the future health or future performance of the animal; a recommendation prioritization engine, where the recommendation prioritization engine is configured to use the predictions to identify an animal or animals at risk for future adverse health or future adverse performance, and to generate one or more recommendations that are prioritized to address the identified risk; and an intervention prioritization engine, where the intervention prioritization engine is configured to use the one or more predictions or recommendations to generate one or more interventions that are prioritized to address the identified risk. The multigenerational microbiome engine further can include a report generation engine configured to receive the predictions, recommendations, or interventions and generate one or more reports that include the specific predictions, recommendations, or interventions, or a combination thereof, and a detailed rationale about adjustments suitable for optimizing the future health or future performance in an animal or group of animals; and an adjustment component configured to implement the one or more interventions as one or more adjustments to the animal or group of animals.

The present disclosure can include a method for predicting future health or future performance in an animal. The method can include obtaining a sample dataset indicative of an entire microbial community within a gastrointestinal tract, mammary gland, or reproductive system, or respiratory system of the animal. The method further can include defining a query including one or more target queries for future health or future performance of the animal. The method further can include selecting a model from a model repository based on the sample dataset and the query including the one or more targets, where each model selected from the model repository is generated by a model generation engine, and wherein the model generation engine has been trained using one or more of metagenomics data or metadata obtained from one or more observational and interventional studies. The method further can include executing the selected model using the sample dataset and the query including the one or more targets to make predictions, generate recommendations, or generate interventions about future health or future performance of the animal. The method further can include reporting the predictions, recommendations, or interventions about future health or future performance, or combinations thereof. The method further can include implementing one or more interventions as one or more adjustments to the animal's nutrition, management system, health care, or rearing environment based on the predictions, recommendations, or interventions.

The present disclosure includes a method for optimizing future health or future performance in an animal. The method can include obtaining a sample dataset indicative of an entire microbial community within a reproductive system of the animal. The method further can include defining a query including one or more target queries for future health or future performance of the animal. The method further can include selecting a model from a model repository based on the sample dataset and the query including the one or more targets. The method further can include executing the selected model using the sample dataset and the query including the one or more targets to make predictions, generate recommendations, or generate interventions about future health or future performance of the animal. The method further can include reporting the predictions, recommendations, or interventions about future health or future performance, or combinations thereof. The method further can include implementing one or more interventions as one or more adjustments to the animal's nutrition, management system, health care, or rearing environment based on the predictions, recommendations, or interventions.

The present disclosure includes a method for optimizing future health or future performance in an animal. The method can include obtaining a sample dataset indicative of an entire microbial community within a mammary gland of the animal. The method further can include defining a query including one or more target queries for future health or future performance of the animal. The method further can include selecting a model from a model repository based on the sample dataset and the query including the one or more targets. The method further can include executing the selected model using the sample dataset and the query including the one or more targets to make predictions, generate recommendations, or generate interventions about future health or future performance of the animal. The method further can include reporting the predictions, recommendations, or interventions about future health or future performance, or combinations thereof. The method further can include implementing one or more interventions as one or more adjustments to the animal's nutrition, management system, health care, or rearing environment based on the predictions, recommendations, or interventions.

The present disclosure provides a method for optimizing future health or future performance in an animal. The method can include obtaining a sample dataset indicative of an entire microbial community within a respiratory system of the animal. The method further can include defining a query including one or more target queries for future health or future performance of the animal. The method further can include selecting a model from a model repository based on the sample dataset and the query including the one or more targets. The method further can include executing the selected model using the sample dataset and the query including the one or more targets to make predictions, generate recommendations, or generate interventions about future health or future performance of the animal. The method further can include reporting the predictions, recommendations, or interventions about future health or future performance, or combinations thereof. The method further can include implementing one or more interventions as one or more adjustments to the animal's nutrition, management system, health care, or rearing environment based on the predictions, recommendations, or interventions.

### BRIEF DESCRIPTION OF THE FIGURES

The drawings illustrate generally, by way of example, but not by way of limitation, various aspects discussed herein.

FIG. 1 is a schematic representation of a multigenerational microbiome system in accordance with various aspects herein.

FIG. 2 is a schematic representation of the components of a multigenerational microbiome system in accordance with various aspects herein.

FIG. 3 is a schematic representation of a model selection matrix in accordance with various aspects herein.

FIG. 4 is a schematic representation of a model creation system in accordance with various aspects herein.

FIG. 5 is a schematic representation of a process flow in accordance with various aspects herein.

FIG. 6 is a flow diagram of a method in accordance with various aspects herein.

FIG. 7 is a schematic representation of a computing environment in accordance with various aspects herein.

### DETAILED DESCRIPTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

The pig microbiome has a strong correlation to an animal's future health and performance. The microbiome of the pig can be used to understand and predict future health and performance throughout any stage of the pig lifespan. The microbiome of the pig can be sampled at any number of locations from the pig, including the gastrointestinal tract, the reproductive system, mammary glands, and the respiratory system, and can be used to extract metagenomic data useful in identifying relationships between the current state of the microbiome and future health and performance outcomes. The present disclosure provides use of multigenerational microbiome data to predict future health and future performance measures in animals. The microbiome of a given animal or group of animals can be repeatedly investigated during the lifespan of the animal to provide custom interventions for optimizing the future health and future performance measures of that animal or group of animals. The systems and methods described herein utilize multiple predictive models for selecting high-performing animals and/or for identifying areas where low-performing animals require custom interventions to improve their performance. The systems and methods herein also utilize multiple predictive models for selecting animals exhibiting robust microbiome and/or for identifying areas where animals with general health concerns are selected for custom interventions to improve their health.

As used herein, the term “gastrointestinal tract” can refer to the tract or passageway that contains all of the major organs of the digestive system (e.g., the mouth, the esophagus, the stomach, the small intestine, and the large intestine and that leads from the mouth to the anus.

As used herein, the “reproductive system” can refer to the tissues, glands, and organs involved in producing offspring. In particular, the reproductive system of the female animals described herein (e.g., sows) can include the ovaries, the uterine hom, the uterine body, the cervix, the vagina, the vulva, the mammary glands, and the teats.

As used herein, the “respiratory system” can refer to the organs and structures that allow for respiration to facilitate gas exchange within an animal. The organs and structures of the respiratory system can include the nostrils, the nasal passages, the nasal septum, the pharynx, the larynx, the trachea, the bronchial tree, the bronchi, the alveoli, and the lungs.

### Multigenerational Microbiome Systems

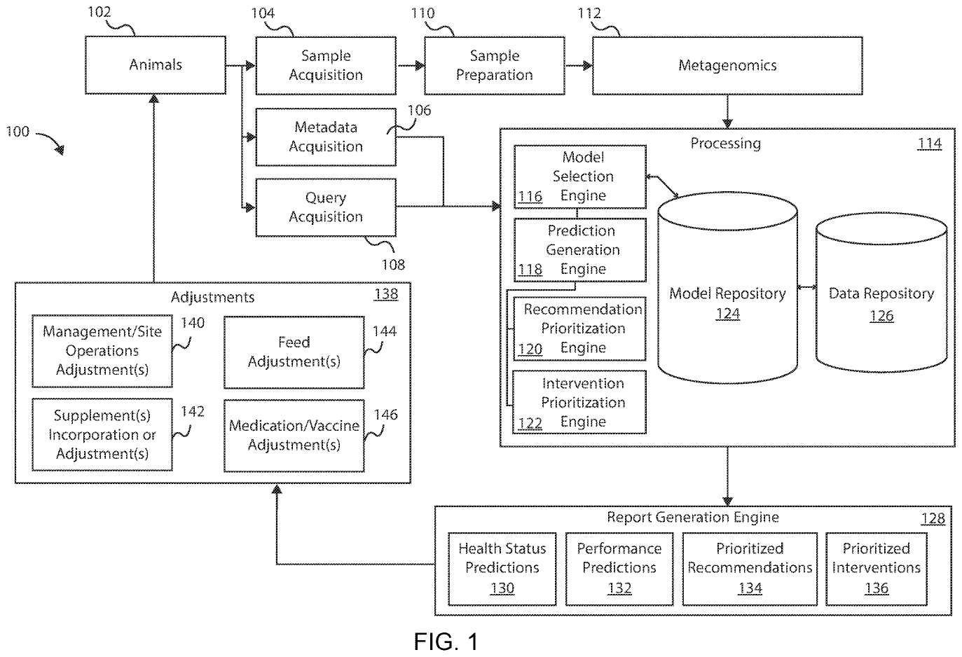

The multigenerational microbiome systems herein can include a number of components configured to exploring the relationship between metagenomics data, pig metadata, and one or more questions to be answered about the future health and performance of an animal or group of animals. Referring now to FIG. 1, a schematic diagram illustrating a multigenerational microbiome system 100 for optimizing future health measures or future performance measures in an animal is shown in accordance with the various aspects herein. Multigenerational microbiome system 100 is configured to generate predictions, recommendations, or interventions about future health measures or future performance measures for an animal or group of animals using multigenerational microbiome data obtained from one or more past generations of animals to predict future health and future performance at the individual animal level or group level. It will be appreciated that the multigenerational microbiome data can include microbiome data obtained from related past generations or from unrelated past generations. The future health measures or future performance measures targeted by the systems and methods herein will be described in more detail elsewhere herein. The multigenerational microbiome data can include metagenomics data obtained from a microbiome sample of any of the current or past generations of animals or groups of animals.

The multigenerational microbiome system 100 can implement any of the many permutations of the methods and techniques as described herein. The multigenerational microbiome system 100 can include an animal component 102, a sample acquisition component 104, a metadata acquisition component 106, a query acquisition component 108, a sample preparation component 110, a metagenomics component 112, a processing component 114, a report generation engine 128, and an adjustments component 138.

As used herein, the term “metadata” can refer to any characteristic about an animal or about how the animal is reared and about any microbiome sampling characteristics, including body weight, birth weight, animal breed, animal sex, body composition, growth rate, feed conversion ratio, mortality, morbidity, livability, illness history, health and performance measures, reproductive measures, current or prior disease states, pathogen risk (e.g., gastrointestinal pathogen risk, respiratory pathogen risk, reproductive system pathogen risk, mammary glands pathogen risk), feed type, vaccinations administered and date of vaccination administration, supplement type, use of anti-microbial resistance promoters, management system (e.g., conventional rearing or rearing animals raised with antibiotics), geographical location, rearing conditions, animal life stage (e.g., piglet, sow, or gilt), microbiome sample type (e.g., fecal, rectal, vaginal, mammary, nasal, oral, lung, etc.), microbiome sampling life stage (e.g., pre-weaning, weaning, post-weaning, grow-finish, gestating, pre-farrowing, farrowing, post-farrowing, postpartum, etc.), microbiome sampling age/time, metagenomics method used (e.g., shotgun DNA-based or RNA-based sequencing, 16S ribosomal RNA gene sequencing, 18S ribosomal RNA gene sequencing, or internal transcribed spacer (ITS) amplicon sequencing), farm location, herd size, animal heart girth measurement, nutrition, and any other data such as demographic or biometric data relevant to an animal or group of animals.

It will be appreciated that the microbiome sample isolated from an animal and used to generated metagenomics data can be isolated from the gastrointestinal tract, including a sample from within the gastrointestinal tract, including but not limited to, oral, cecum, colon, or rectal samples, or a fresh fecal sample; the reproductive system, (e.g., proximal or distal vaginal samples); the mammary glands (e.g., milk or colostrum samples): or the respiratory system, (e.g., nasopharyngeal, bronchoalveolar lavage, or lung samples).

As used herein, the term “metagenomics data” can refer to sequencing data obtained about a microbiome sample, including any of the above-mentioned samples such as a fecal sample, a rectal sample, a vaginal sample, a mammary sample, an oral sample, a nasal sample, or a lung sample using one or more sequencing technologies, including but not to be limited to, whole genome sequencing techniques (e.g., shotgun DNA-based or RNA-based sequencing), 16S ribosomal RNA gene sequencing, 18S ribosomal RNA gene sequencing, or internal transcribed spacer (ITS) amplicon sequencing.

The animal component 102 suitable for the systems and methods described herein can include one or more types of wild or domesticated swine at various life stages, including but not to be limited to piglets, gilts, sows, baconers, barrows, boars, dams, feeders, growers, finishers, pigs, porkers, runts, sires, stags, or hogs. In various aspects, the animal component 102 suitable for the systems and methods described herein can include piglets, gilts, or sows. The animal component 102 further can have associated therewith any additional information about the physical attributes of the animals, including but not to be limited to breed, body weight, body composition, growth rate, feed conversion ratio, mortality, morbidity, livability, illness history, general health, and the like. The animal component 102 further can include techniques, methods, and devices for acquiring any additional information about the animals.

The sample acquisition component 104 can include various systems or techniques for acquiring biological samples from animals. The sample acquisition component 104 can be configured to obtain the biological samples from the animals from one or more farm sites across one or more geographical regions. By way of example, the biological samples can be obtained using a permeable material or substrate, such as swab, sponge, or other material that is configured to wipe and secure biological material (e.g., fluids such as chyme or excreta such as feces from the animals) from a surface or orifice of the gastrointestinal tract of the animals. In another example, the biological samples can be taken using a non-permeable material, such as a glass or polymer tube, vial, or other container that is configured to receive the biological samples directly from the animals or indirectly from the animals such as a fecal sample obtain directly from feces or other excreta. The biological sample can include a sample of the microbial community obtained from within a gastrointestinal tract, reproductive system, mammary glands, or respiratory system of the animals as described elsewhere herein. In an example, the biological sample can be obtained from a segment of the gastrointestinal tract of the animals, such from the stomach, duodenum, jejunum, ileum, cecum, or colon of processed animals. In another example, the biological samples can be obtained from exposed orifices of animals or from droppings produced by the animals. In yet another example, the biological samples can be obtained from the reproductive system, such as from the proximal or distal vagina. In some examples, the biological sample can be obtained from the mammary glands, to include milk or colostrum samples. In other examples, the biological sample can be obtained from the respiratory system, to include nasopharyngeal, bronchoalveolar lavage, or lung samples.

The sample acquisition component 104 can include a standardized sample acquisition assembly (e.g., a sample kit) that includes a glass or polymer tube, a chemical solution or reagent, and one or more swabs or other substrate for retrieving a biological sample. The tube can be pre-filled with a chemical solution. In example, the chemical solution can include a solution that is configured to lyse microbiota cells and preserve DNA and/or RNA. In an example, the sample acquisition assembly can include prescribed sample collection acquisition and handling protocols. Such protocols can include directions regarding the number of samples and timeline for sample collection per animal or group of animals, a process for collecting and storing each sample and a process for shipping the samples for analysis. The sample acquisition assembly, including the described protocols, can standardize the sample acquisition process and thereby reduce variations in the metagenomic data obtained from the samples.

The metadata acquisition component 106 can include various systems or methods for acquiring metadata about animals. The metadata acquisition component 106 can be configured to receive metadata from one or more animals from one or more farm sites across one or more geographical regions. The metadata can include, but is not to be limited to, one or more of body weight, breed, body composition, growth rate, feed conversion ratio, mortality, morbidity, livability, illness history, general health, reproductive measures, disease states, pathogen risk (e.g., gastrointestinal pathogen risk, future respiratory pathogen risk, future reproductive system pathogen risk, future mammary glands pathogen risk), feed data, supplement data, or other data that describes a characteristic of the animal as described elsewhere herein. The metadata acquisition component 106 can be configured to receive input indicative of one or more types of metadata.

The query acquisition component 108 can include various systems or methods for receiving a target query about animals. The query acquisition component 108 can be configured to receive one or more target queries for future health measures or future performance measures of one or more animals. The input received by the query acquisition component 108 can be indicative of a target query selection, a particular type of sequencing data (described elsewhere herein), and the like. The target queries can be generalized to all animal life stages or can be specific to one or more life stages including to the piglet, sow, or gilt life stages. For example, it can be desirable to interrogate one or more target queries for piglets, where the one or more queries investigate any one of piglet growth rate, piglet average daily weight gain, piglet microbiome alpha diversity, piglet livability, piglet mortality, piglet general health, piglet gastrointestinal pathogen risk, piglet respiratory pathogen risk, piglet anti-microbial growth promoter use, piglet microbiome composition, piglet microbiome function, piglet microbiome network interaction, piglet microbiome stability, piglet microbiome robustness, and/or piglet microbiome resilience. It will be appreciated that the target queries for future health measures or future performance measures of piglets can include one or more current or future life stages of the piglet, such as birth, a nursery stage, a pre-weaning stage, a weaning stage, a post-weaning stage, a grow-finish stage, or during any transition between any of the forgoing from piglet to adult pig.

It further can be desirable to interrogate one or more target queries for sows, where the one or more queries investigate any one of sow livability, sow mortality, sow general health, sow gastrointestinal pathogen risk, sow respiratory pathogen risk, sow reproductive system pathogen risk, sow mammary glands pathogen risk, sow anti-microbial growth promoter use, sow microbiome composition, sow microbiome function, sow microbiome network interaction, sow microbiome stability, sow microbiome robustness, sow microbiome resilience, sow reproductive performance, and/or sow to piglet ratios.

It further can be desirable to interrogate one or more target queries for gilts, where the one or more queries investigate any one of gilt livability, gilt mortality, gilt general health, gilt gastrointestinal pathogen risk, gilt respiratory pathogen risk, gilt reproductive system pathogen risk, gilt anti-microbial growth promoter use, gilt microbiome composition, gilt microbiome function, gilt microbiome network interaction, gilt microbiome stability, gilt microbiome robustness, gilt microbiome resilience, and/or gilt future reproductive performance.

The sample preparation component 110 can include any suitable system or technique for preparing a biological sample obtained from animals for digitization, such as for generating metagenomic data on the biological samples. The sample preparation component 110 can include preparation of a sample from the gastrointestinal tract, reproductive system, mammary glands, or respiratory system of the animal, as described elsewhere herein, in order to obtain metagenomic data about the microbiome of the animal from such locations. In the case of microbiome data, the sample can be prepared for obtaining metagenomic data using sequencing that provide sequences (e.g., deoxyribonucleic acid (DNA) sequences or ribonucleic (RNA) acid sequences) such as operational taxonomic unit (OTU) sequences, amplicon sequence variants (ASV), 16S ribosomal RNA gene sequences, 18S ribosomal RNA gene sequences, shotgun sequences, internal transcribed spacer (ITS) amplicon sequences, or any other suitable genetic marker sequences. The metagenomic data can include information that is indicative of the relative or absolute abundance, diversity, taxonomy, or distribution of microbiota of given taxonomic ranks in the microbiome of the animal from which the biological sample is obtained. In certain examples, the metagenomic data can include data that is indicative of metabolites detected in the biological sample, such as to identify functional aspects of a microbiome, including selected metabolic pathways or catalytic activity.

The metagenomics component 112 can include any suitable analysis techniques and tools for generating metagenomics data. The metagenomics component 112 can be configured to receive metagenomics data obtained from a microbiome sample of one or more animals. The metagenomics component 112 can apply any number of techniques and tools to directly or indirectly assess the genetic content of a sample of the microbial community obtained from within a gastrointestinal tract or reproductive system of the animals. The metagenomics component 112 can generate information about the functional gene composition and/or nucleotide variations within the sample of the microbial community. The metagenomics component 112 can apply one or more sequencing technologies to a microbiome sample, including but not to be limited to, whole genome sequencing techniques including shotgun sequencing, 16S ribosomal RNA gene sequencing, 18S ribosomal RNA gene sequencing, or internal transcribed spacer (ITS) amplicon sequencing, and the like. It will be appreciated that when analyzing samples of the microbial community prepared from the gastrointestinal tract, reproductive system, mammary glands, or respiratory system, the entirety of the metagenomic data representative of those samples is analyzed without further parsing of the data into a predetermined subset of metagenomic data. By way of example, the metagenomics data generated by the systems and methods herein includes the entire 16S RNA gene sequence, the entire 18S RNA gene sequence, the entire shotgun metagenome, and the like. Such metagenomics data are not further reduced to a smaller subset of data. It will further be appreciated that the metagenomics data generated by the systems and methods herein are not pooled at any time during the acquisition, preparation, or analysis of the samples. Further, it will be understood that the samples of the microbial community analyzed herein are unique to each individual animal being investigated.

The processing component 114 can be configured to receive metadata, target queries, and metagenomics data from the metadata acquisition component 106, the query acquisition component 108, and the metagenomics component 112. The processing component 114 can include any number of computing resources including one or more of a computer, a tablet, a mobile telephone, a server, a computing system, a microprocessor, circuitry, memory, a computing environment or a partition of a computing environment, all of which are allocated to a user of the computing resources and that are configured to process metagenomic data. The processing component 114 can be coupled to a communications network such as the Internet, a wireless network a local area network (LAN), a wide area network (WAN), and the like, and it can be configured to communicate via wireless communications including wi-fi, Bluetooth, satellite, and the like, or via wired connections including telephone, coaxial cable, fiber-optic, twister pair cables, and the like.

The processing component 114 further can include a one or more machine learning components, including a model selection engine 116, a prediction generation engine 118, a recommendation prioritization engine 120 or an intervention prioritization engine 122. The processing component 114 further can include a model repository 124 and a data repository 126. It will be appreciated that the processing component 114 further can include additional components not shown in FIG. 1, including a model generation engine, a model update engine, and the like.

The model selection engine 116 can be configured to select one or more trained models from model repository 124 based on a sample data set and a target query. The sample data set can include one or more types of data, including metadata and/or metagenomics data, as defined herein. The model selection engine 116 can be configured to select a model from the model repository 124 that best fits the inputs received. By way of example, for every combination of metadata, metagenomics data and type, and selected target query, the model selection engine 116 can be configured to select one or more trained and validated models from the model repository 124 that are configured to provide one or more predictions, recommendations, or interventions to answer the user defined query. It will be appreciated that model selection engine 116 provides the model or set of models to the prediction generation engine 118 once a model or set of models is selected. Exemplary models are described in more detail elsewhere herein.

The prediction generation engine 118 can be configured to execute a model or set of models selected by model selection engine 116. Execution of the models can include supplying the model with the entirety of the metagenomics data to generate one or more predictions, which can be qualitative or quantitative, and that pertain to the query specific to the model and can relate to the future health measures or future performance measures of the animal. Each model executed by the prediction generation engine 118 can generate a different set of predictions. The predictions generated by the prediction generation engine 118 can be provided to the recommendation prioritization engine 120 and to the intervention prioritization engine 122 where they are used to identify risk and prioritize solutions.

The recommendation prioritization engine 120 can be configured to use the predictions generated by the prediction generation engine 118 to identify an animal or animals that are at risk for future adverse health or future adverse performance and generate one or more recommendations that are prioritized to address the identified risk. By way of example, the recommendations can be tailored for improving the future health measures or the future performance measures of an animal or group of animals in view of the original target query. Exemplary recommendations are described in more detail elsewhere herein.

The intervention prioritization engine 122 can be configured to use the one or more recommendations identified and prioritized by the recommendation prioritization engine 120 to identify an animal or animals that are at risk for future adverse health or future adverse performance and/or generate one or more interventions that are prioritized to address the identified risk. By way of example, the interventions can be tailored for improving the future health measures or the future performance measures of an animal or group of animals in view of the original target query. Exemplary interventions are described in more detail elsewhere herein.

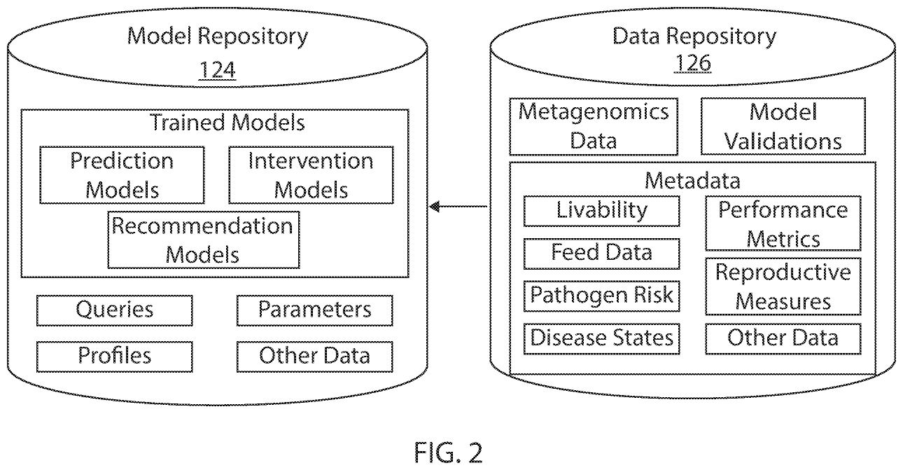

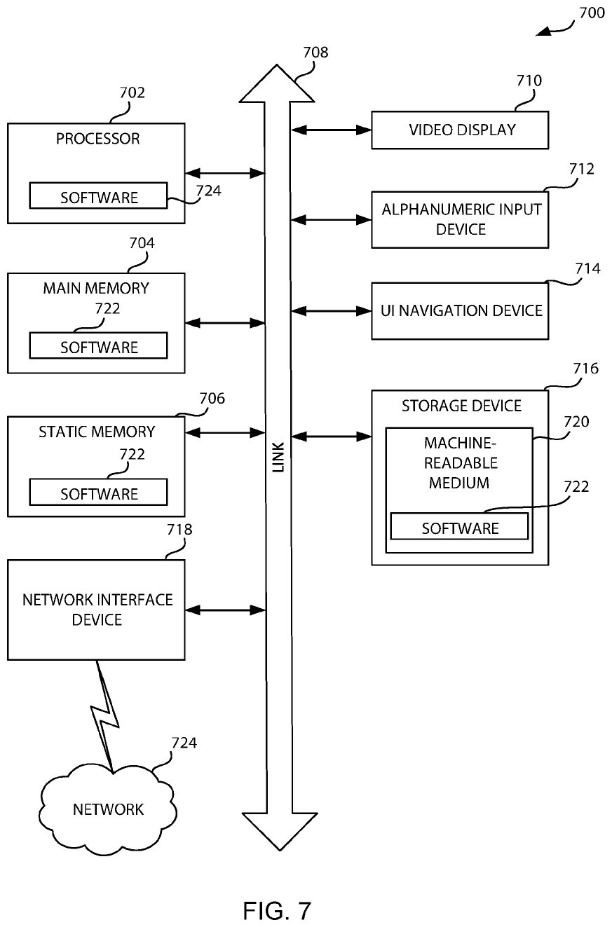

The processing component 114 can further include a model repository 124 and a data repository 126. Referring now to FIG. 2, the model repository 124 and the data repository 126 are shown in more detail in accordance with various aspects herein. The model repository 124 can be configured to store one or more trained and validated models. The models can include one or more models including prediction models, recommendation models, or intervention models. The models stored in the model repository 124 include models that are identified by their associated parameter data. The model repository 124 further can be configured to store the parameter data, where the parameter data can be specific to a given model to differentiate one model from another, where the parameter data correlates with the metadata as defined herein. Once a target query is defined, the model selection engine 116 can look to the parameters to select the appropriate model. The model repository 124 further can be configured to store profile data, where the profile data can include profiles used previously that are used to compare new predictions and to determine how to prioritize a given recommendation or intervention. The models can be stored in memory, such as in RAM. DRAM, SRAM. ROM, PROM, EPROM, EEPROM, etc., and as discussed in more detail in reference to FIG. 7. The models can be stored locally, or they can be stored on a network, such as within cloud computing resources or various servers or databases that are communicatively coupled to processing component 114. In various aspects the model repository 124 is preestablished prior to obtaining the predictions, recommendations, and interventions, as will be described with respect to training the models elsewhere herein. In various aspects the model repository 124 is a database.

The data repository 126 can be configured to store various types of data including metagenomics data and metadata. The metagenomics data can include sequences obtained using whole-genome sequencing, 16S ribosomal RNA gene sequences, 18S ribosomal RNA gene sequences, shotgun sequences, internal transcribed spacer (ITS) amplicon sequences, or any other suitable genetic marker sequences. The metadata can include one or more of a model number, animal life stage (e.g., piglet, sow, or gilt), sample type, sampling time (e.g., pre-weaning, weaning, post-weaning, grow/finish, gestation, pre-farrowing, farrowing, post-farrowing, etc.), metagenomics method used, animal breed, farm location, herd size, management system, animal weight, animal heart girth measurement, feed data, livability, pathogen risk (e.g., gastrointestinal pathogen risk, future respiratory pathogen risk, future reproductive system pathogen risk, future mammary glands pathogen risk), disease states, performance metrics, reproductive measures, and any other data such as demographic or biometric data relevant to an animal or group of animals. The data repository 126 further can include one or more model validations, where the model validations include model validation data related to the performance of the models, including but not to be limited to model accuracy, model sensitivity, etc. Model validation data can also be used by model selection engine 116 when selecting the model (e.g., prediction model, recommendation model, or intervention model) having the best fit. The data repository 126 can be communicatively coupled to the model repository 124. The metagenomics data and metadata can be stored in memory, such as in RAM, DRAM, SRAM, ROM, PROM, EPROM, EEPROM, etc., and as discussed in more detail in reference to FIG. 7. The metagenomics data and metadata can be stored locally, or can be stored on a network, such as within cloud computing resources or various servers or databases that are communicably coupled to the processing component 114. In various aspects the data repository 126 can be a database.

The report generation engine 128 can be configured to receive the predictions, recommendations, and interventions from the processing component 114 to generate one or more reports that include the specific predictions, recommendations, or interventions, or a combination thereof, and a detailed rationale about adjustments suitable for optimizing the future health measures or future performance measures in an animal or group of animals. It will be appreciated that the report generation engine 128 can be integral to the processing component 114 or can be a separate component. The reports generated by report generation engine 128 can include one or more health status predictions 130, performance predictions 132, prioritized recommendations 134, and prioritized interventions 136. In an aspect, the report generation engine 128 can be configured to generate one or more reports in a computer readable data structure for display on a graphical user interface that is configured to provide a user with the report, including the specific predictions, recommendations, or interventions and associated rationale as tailored to a specific animal. The one or more reports can be communicated by the report generation engine 128 or by processing component 114 to a user display, which can be static or interactive, and can further provide information about purchasing customized feed or customized supplements to improve the future health measures or future performance measures of an animal or group of animals. The reports generated by the report generation engine 128 can be used to guide the implementation the one or more predictions, recommendations, or interventions in the field. It will be appreciated that the predictions, recommendations, or interventions are configured to reduce the incidence or seventy of disease, improve health and performance measures in the animal, reduce the number of animals needing to be culled from a population, select individual animals including an indication for enhanced future health measures or future performance measures, identify individual animals that require interventions, or reduce the reliance on antimicrobial medications.

The adjustments component 138 can be configured to implement one or more interventions that are generated and prioritized by the intervention prioritization engine 122 as one or more adjustments to an animal or group of animals' nutrition, management system, health care, or rearing environment. The management system can include a system of conventional animal rearing, such as with the use of antimicrobial compounds as prophylaxis (e.g., antibiotics, zinc oxide, etc.), or a system of rearing where the animals are raised using altematives to antimicrobial compounds (e.g., prebiotics, probiotics, postbiotics, phytogenics, etc.). The rearing environment can include farm location, herd culling, and the like. The adjustments can include one or more management and/or site operations adjustments 140 at the farm, including but not to be limited to, switching from conventional rearing to an environment where the animals are raised without antimicrobials, or vice versa, increasing or decreasing herd size, implementing one or more culling decisions, isolating one or more animals, and the like. The adjustments can include implementing one or more supplement incorporations or adjustments 142, including, but not to be limited to, adding or removing one or more supplement compositions to address one or more nutrition deficiencies or to optimize gastrointestinal, reproductive, mammary, or respiratory measures, changing an existing supplement composition, adding or removing a supplement containing one or more anti-microbial growth promoters into the diet, and the like. The adjustments can include making one or more feed adjustments 144, including, but not to be limited to, adding or removing a feed composition or compositions to address one or more nutrition deficiencies, changing a feed composition such as switching from a nursery feed to an adult feed, adding of one or more vitamins or minerals, adding or removing one or more anti-microbial growth promoters into a feed, adding or removing prebiotics, probiotics, postbiotics, phytogenics, and the like. The adjustments can include making one or more medication or vaccine adjustments 146, including, but not to be limited to, administering one or more vaccines to prevent or treat a disease, administering one or more medications to prevent or treat a disease, and the like.

### Target Queries

The target queries herein can define a question about the future health or the future performance of an animal or group of animals. The target queries can include any one of a number of questions configured to predict a future health measure or future performance measure in an animal. The target queries can include an inquiry on any one of the following non-limiting examples for any one of a piglet, a sow, or a gilt, or groups thereof, where developmentally appropriate: future birth weight, future body weight, future feed conversion ratio, future feed intake, future body composition, future growth rate, future average daily weight gain, future heart girth diameter, future livability, future mortality, future morbidity, future gastrointestinal pathogen risk, future respiratory pathogen risk, future reproductive system pathogen risk, future mammary glands pathogen risk, future litter size, future number of liveborn piglets, future number of stillborn piglets, future parity, future sow mortality, or future incidence of uterine prolapse, or any combination thereof.

### Models

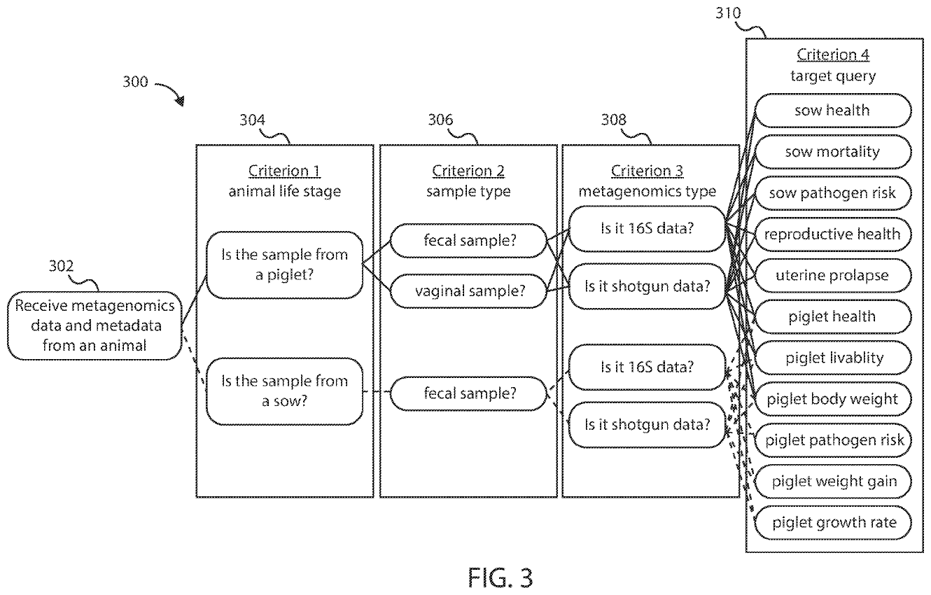

The models herein can be selected by the model selection engine 116 as described above. In an aspect, model selection by the model selection engine 116 can occur according to one or more model selection matrices. Referring now to FIG. 3, an exemplary model selection matrix 300 is shown in accordance with various aspects herein. The model selection matrix 300 can be configured to receive metagenomics data and metadata at 302. The model selection matrix 300 analyzes the metagenomics data and metadata and determines from what life stage of animal the microbiome sample data was obtained according to criterion 1 at 304, where this can further include the gilt stage (not shown). The model selection matrix 300 analyzes the metagenomics data and metadata and determines what type of sample the microbiome sample data represents according to criterion 2 at 306. It will be appreciated that while only the fecal sample or vaginal sample are shown at 306, the other types of biological samples described herein can also be considered at this step. The model selection matrix 300 analyzes the metagenomics data and metadata and determines what type of metagenomics analysis was performed on to obtain the metagenomics data according to criterion 3 at 308. Multiple types of metagenomics data are defined elsewhere herein and are further considered at 308. The model selection matrix 300 analyzes the target query and then determines a model or set of models suitable for providing a prediction based on the inputs according to criterion 4 at 310. It will be appreciated that not all target queries, as described herein, are listed at 310, but are withing the scope of the model selection matrix 300 as described.

The models herein can include trained models for the prediction of health and performance, prediction of optimal recommendations for nutrition and health management, and the prediction of most appropriate and effective interventions to implement with the animals. The models herein can include trained models including prediction models, recommendation models, and intervention models. The models can be trained according to one or more supervised or unsupervised machine learning algorithms, microbial network modeling, and the metagenomics data and metadata obtained from one or more observational swine studies or interventional swine studies and a given target query. Machine learning algorithms suitable for use herein can include different artificial intelligence/machine learning such as supervised or unsupervised machine learning algorithms. Examples of suitable supervised machine learning algorithms can include, but are not to be limited to, Bayesian networks, decision trees, K-nearest neighbors, linear classifiers, linear regression, logistic regression, naïve Bayesian algorithms, neural networks, quadratic classifiers, random forests, support vector machines (SVMs), XGBoost, and other suitable algorithms. Examples of suitable unsupervised machine learning algorithms include, but are not to be limited to, expectation-maximization algorithms, vector quantization, information bottleneck methods, k-means clustering, hierarchical clustering, and dimensionality reduction (e.g. principal component analysis).

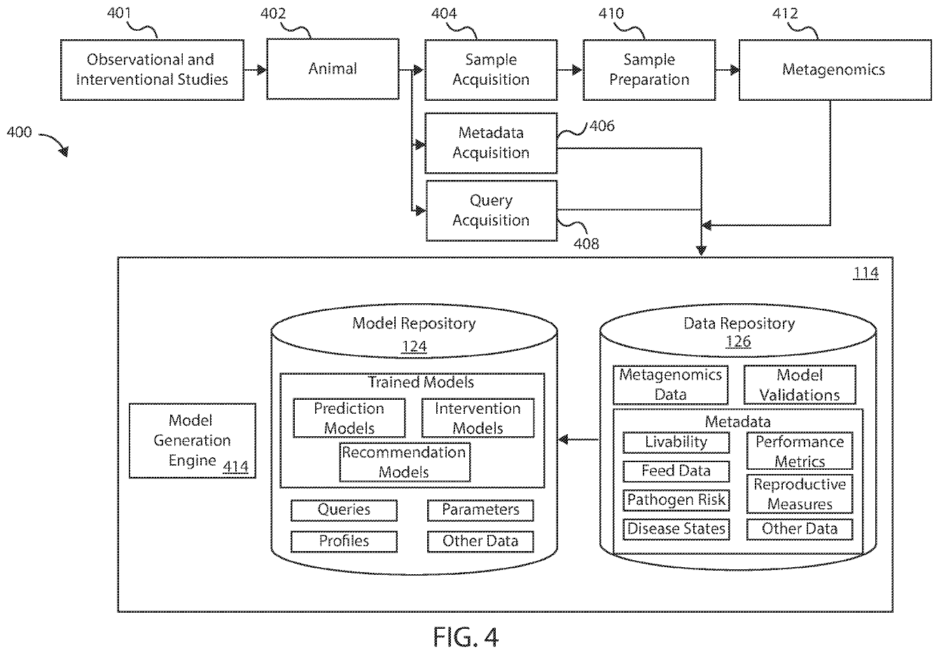

Referring now to FIG. 4, a schematic diagram illustrating a model creation system 400 for creating a repository of models is shown in accordance with the various aspects herein. The model creation system 400 can include analogous components configured to operate as described with respect to multigenerational microbiome system 100 in FIG. 1, including animal component 402, sample acquisition component 404, metadata acquisition component 406, query acquisition component 408, sample preparation component 410, and metagenomics component 412. The model creation system 400 further can include one or more observational studies and interventional studies 401. The model creation system 400 can be communicatively coupled with the processing component 114 of multigenerational microbiome system 100 such that the models generated by the model creation system 400 can be deposited and stored in model repository 124. The processing component 114 further can include a model generation engine 414, where model generation engine 414 is configured to receive metagenomics data, metadata, and a target query from one or more observational studies and interventional studies 401 to generate the models suitable for use herein. It will be appreciated that the models herein can be continually improved and updated by using data obtained from additional observational studies and interventional studies, as well as by using data obtained from application of the systems and methods herein at a one or more production farm locations where the animals are being reared for market.

The observational studies and interventional studies 401 can be used to provide multigenerational microbiome data obtained from one or more past generations to predict future health and future performance at the individual animal level or group level. The observational swine studies can include those studies conducted by observing piglets, sows, and gilts in their rearing environment and collecting data about various health and performance metrics across multiple generations of animals. The observational studies are conducted without interference, such as by controlling the environment, feed rations, medication schedules, culling, vaccination schedules, and the like. The interventional studies can include those studies conducted by controlling one or more variables and performing one or more interventions to obtain data on how animals respond on the individual or group level, as well as across generations. Data obtained from observational studies and interventional studies are used to train the models herein such as by identifying one or more health or performance risks in need of intervention to optimize future health measures or future performance measures of an animal or group of animals. The trained models are then incorporated into model repository 124 for use in generating one or more predictions, recommendations, or interventions based on the input target query, metadata, and metagenomics data.

The model validations can include information to validate any of the prediction models, the recommendation models, or the intervention models stored in the data repository 126. The model validations can include a process or processes executed after training a given model to confirm that the model achieves its intended purpose. In an aspect, the model validations can include determining the predictive accuracy of a given model.

The prediction models can include those models that include one or more predictions regarding the future health measures or future performance measures of an animal. The prediction models can include, but are not to be limited to one or more prediction model that: predict future body weight, future feed conversion ratio, future feed intake, future body composition, future growth rate, future average daily weight gain, future heart girth diameter, future livability, future mortality, future morbidity, future gastrointestinal pathogen risk, future respiratory pathogen risk, future reproductive system pathogen risk, future mammary glands pathogen risk, future litter size, future number of liveborn piglets, future number of stillborn piglets, future parity, future sow mortality, or future incidence of uterine prolapse, or any combination thereof. It will be appreciated that in some aspects of the methods herein, the predictions are provided to a user for prognostic purposes.

The recommendation models can include those models that include one or more recommendations regarding the future health measures or future performance measures of an animal. The recommendation models can include, but are not to be limited to one or more recommendation models that recommend to: change a feed or supplement composition, add or remove a feed or supplement composition, administer one or more vaccines, administer one or more medications, make alterations to a rearing environment or management system, select an animal including an indication for enhanced future health measures or future performance measures, culling an animal including an indication for diminished future health measures or future performance measures, select a gilt including an indication for enhanced future health measures or future performance measures, or add or remove anti-microbial growth promoters into a diet of the animal, or any combination thereof. It will be appreciated that in some aspects of the methods herein, the predictions and recommendations are provided to a user along with customized management recommendations.

The intervention models can include those models that include one or more interventions regarding the future health measures or future performance measures of an animal. The intervention models can include, but are not to be limited to one or more intervention models that provide interventions including: changing a feed or supplement composition, supplying a feed or supplement composition, adding or removing a feed or supplement composition, administering one or more vaccines, administering one or more medications, making alterations to the rearing environment and management system, making alterations to the management system, selecting an animal including an indication for enhanced future health measures or future performance measures, culling an animal including an indication for diminished future health measures or future performance measures, selecting a gilt including an indication for enhanced future health measures or future performance measures, or adding or removing one or more anti-microbial growth promoters into a diet of the animal. It will be appreciated that in some aspects of the methods herein, the interventions are provided to a user along with providing customized management interventions including customized feed or supplement interventions.

The models herein can include one or more models for generating predictions, recommendations, or interventions for predicting the future health measures or future performance measures of an animal, where the animal is a swine animal, and where the swine animal is a piglet, a sow, or a gilt. In an aspect, the models herein can include one or more piglet models, one or more sow models, or one or more gilt models. In an aspect, the models include one or more piglet models. In an aspect, the models include one or more sow models. In an aspect, the models include one or more gilt models.

### Piglet Models

The piglet models can include models for predicting future health measures or future performance measures in piglets using metagenomics data obtained from a microbiome sample from a piglet, including one or more piglet fecal samples or piglet rectal samples, or any other biological sample from the piglet as described elsewhere herein. The piglet models can be used to predict health measures or performance measures at a future time point in the piglet's life or a group of piglets' lives, including one or more life stages such as birth, a nursery stage, a pre-weaning stage, a weaning stage, a post-weaning stage, a grow-finish stage, or during any transition between any of the forgoing from piglet to adult pig.

By way of non-limiting example, the following piglet models are contemplated for use in the systems and methods described herein, for any of the life stages of the piglet (e.g., birth, pre-weaning, weaning, post-weaning, transitioning from piglet to adulthood, or during any transition between any of the forgoing):

Piglet growth rate model: This model has been trained and validated for use in predicting the growth rate of one or more piglets, either qualitatively (e.g. in bins high/low, quartiles, above/below median etc.) or quantitatively at a future time point in the piglet's production life.

Piglet average daily gain model: This model has been trained and validated for use in predicting the average daily gain of body weight of one or more piglets, either qualitatively (e.g. in bins high/low, quartiles, above/below median etc.) or quantitatively at a given time, from within a range of a given time, or during a future time point of the piglet's life.

Piglet alpha diversity model: This model has been trained and validated for use in predicting the alpha diversity of the gut microbiome of one or more piglets at specific life stages of the piglet, either qualitatively (e.g. in bins high/low, quartiles, above/below median etc.) or quantitatively.

Piglet mortality model: This model has been trained and validated for use in predicting the mortality (e.g., probability of death) of one or more piglets at specific stages of life, either qualitatively (e.g. in bins high/low, quartiles, above/below median etc.) or quantitatively.

Piglet livability model: This model has been trained and validated for use in predicting the livability (e.g., probability of survival) of one or more piglets at specific stages of life, either qualitatively (e.g. in bins high/low, quartiles, above/below median etc.) or quantitatively.

Piglet general health model: This model has been trained and validated for use in predicting the general health status or health measures of the one or more piglets at specific stages of life qualitatively (e.g. in bins good/bad, high/low, quartiles, etc.) or quantitatively.

Piglet gastrointestinal pathogen risk model: This model has been trained and validated for use in predicting the probability of incidence and/or severity of singular and/or plural gastrointestinal pathogen infection (e.g. including, but not to be limited to, infection due to Escherichia coli, Salmonella enterica, Streptococcus suis, Clostridium perfringens type A and C, Lawsonia intracellularis, Brachyspira hyodysenteriae, Eimeria sp., Isospora suis, and Campylobacter coli, porcine epidemic diarrhea virus, rotavirus) of one or more piglets at specific stages of life, either qualitatively (e.g. in bins high/low, quartiles, above/below median etc.) or quantitatively.

Piglet respiratory pathogen risk model: This model has been trained and validated for use in predicting the probability of incidence and/or severity of singular and/or plural respiratory pathogen infection (e.g. including, but not to be limited to, infection due to Streptococcus suis, Mycoplasma hyopneumoniae, Actinobacillus pleuropneumoniae, Glaesserella parasuis (formerly classified as Haemophilus parasuis), Pasteurella multocida, Bordetella bronchiseptica, Mycoplasma hyorhinis, porcine reproductive and respiratory syndrome virus (i.e., PRRS), influenza, and porcine circovirus type 2/3) of one or more piglets at specific stages of life, either qualitatively (e.g. in bins high/low, quartiles, above/below median etc.) or quantitatively.

Piglet anti-microbial growth promoter model: This model has been trained and validated for use in predicting the use and/or benefit of specific anti-microbial growth promoters for one or more piglets at specific stages of life, either qualitatively (e.g. in bins high/low, quartiles, above/below median etc.) or quantitatively.

Piglet biotic health and growth promoter model: This model has been trained and validated for use in predicting the use and/or benefit of biotic (e.g., prebiotics, probiotics, postbiotics, antibiotics, phytogenics) health and growth promoters for one or more piglets at specific stages of life, either qualitatively (e.g. in bins high/low, quartiles, above/below median etc.) or quantitatively.

Piglet microbiome composition model: This model has been trained and validated for use in predicting the composition of the gut microbiome and associated metrics (such as richness, diversity, or relative or absolute abundances of taxa) of one or more piglets at specific stages of life, either qualitatively (e.g. in bins high/low, quartiles, above/below median etc.) or quantitatively.

Piglet microbiome functional model: This model has been trained and validated for use in predicting the functional potential and/or capacity of the gut microbiome (as measured via the carbohydrate active enzyme activity, and KEGG pathways, amongst others) of one or more piglets at specific stages of life, either qualitatively (e.g. in bins high/low, quartiles, above/below median etc.) or quantitatively.

Piglet microbiome network interaction model: This model has been trained and validated for use in predicting the interactions between the microbial community members, including but not limited to hub species, and positive and negative interactions, in the gut of the one or more piglets at specific stages of life, either qualitatively (e.g. in bins high/low, quartiles, above/below median etc.) or quantitatively.

Piglet microbiome stability model: This model has been trained and validated for use in predicting the stability (as defined by the day-to-day variation in the microbiome) of the microbiome of one or more piglets at specific stages of life, either qualitatively (e.g. in bins high/low, quartiles, above/below median etc.) or quantitatively.

Piglet microbiome robustness model: This model has been trained and validated for use in predicting the robustness (defined by the capacity of microbiome to resist changes caused by stressors) of the microbiome of one or more piglets at specific stages of life, either qualitatively (e.g. in bins high/low, quartiles, above/below median etc.) or quantitatively.

Piglet microbiome resilience model: This model has been trained and validated for use in predicting the resilience (as defined by the capacity and the time it takes for the microbiome to return to equilibrium upon the act of stressors) of the microbiome of one or more piglets at specific stages of life, either qualitatively (e.g. in bins high/low, quartiles, above/below median etc.) or quantitatively.

### Sow Models

The sow models can include models for predicting future health measures or future performance measures in sows using microbiome data obtained from a microbiome sample from a sow, including one or more sow fecal samples, sow rectal samples, sow vaginal samples, sow mammary samples, or sow respiratory samples. The sow models can be used to predict health measures or performance measures at a future time point in the sow's life or a group of sows' lives, including one or more life stages such as gestating a litter, nursing a litter, a time period preparing for the next litter, postpartum, or during any transition between any of the forgoing.

By way of non-limiting example, the following sow models are contemplated for use in the systems and methods described herein for sows at any of the various life stages of a sow (e.g., pre-farrowing, farrowing, post-farrowing, gestating, lactating, postpartum, etc.):

Sow mortality model: This model has been trained and validated for use in predicting the mortality (e.g., probability of death) of one or more sows at specific stages of life, either qualitatively (e.g. in bins high/low, quartiles, above/below median etc.) or quantitatively.

Sow livability model: This model has been trained and validated for use in predicting the livability (e.g., probability of survival) of one or more sows at specific stages of life, either qualitatively (e.g. in bins high/low, quartiles, above/below median etc.) or quantitatively.

Sow general health model: This model has been trained and validated for use in predicting the general health status of one or more sows at specific stages of life qualitatively (e.g. in bins good/bad, high/low, quartiles) or quantitatively.

Sow gastrointestinal pathogen risk model: This model has been trained and validated for use in predicting the probability of incidence and/or severity and/or from carrier/exposure risk perspective of singular and/or plural gastrointestinal pathogen infection (e.g. including, but not to be limited to, infection due to Escherichia coli, Salmonella enterica, Streptococcus suis, porcine epidemic diarrhea virus, Clostridium perfringens type A and C, Lawsonia intracellularis, Brachyspira hyodysenteriae, Eimeria sp., Isospora suis, Campylobacter coli) of one or more sows at specific stages of life, either qualitatively (e.g. in bins high/low, quartiles, above/below median etc.) or quantitatively.

Sow respiratory pathogen risk model: This model has been trained and validated for use in predicting the probability of incidence and/or severity and/or from carrier/exposure risk perspective of singular and/or plural respiratory pathogen infection (e.g. including, but not to be limited to, infection due to Streptococcus suis. Mycoplasma hyopneumoniae, Actinobacillus pleuropneumoniae, Glaesserella parasuis (formerly classified as Haemophilus parasuis), Pasteurella multocida, Bordetella bronchiseptica, Mycoplasma hyorhinis, porcine reproductive and respiratory syndrome virus (i.e., PRRS), influenza, porcine circovirus type 2/3) of one or more sows at specific stages of life, either qualitatively (e.g. in bins high/low, quartiles, above/below median etc.) or quantitatively.

Sow reproductive system pathogen risk model: This model has been trained and validated for use in predicting the probability of incidence and/or severity and/or from carrier/exposure risk perspective of singular and/or plural respiratory pathogen infection (e.g. including, but not to be limited to, infection due to porcine reproductive and respiratory syndrome virus (i.e., PRRS), porcine circovirus type 2/3) of one or more sows at specific stages of life, either qualitatively (e.g. in bins high/low, quartiles, above/below median etc.) or quantitatively.

Sow mammary glands pathogen risk model: This model is used to predict the probability of incidence and/or severity and/or from carrier/exposure risk perspective of singular and/or plural mammary pathogen infection (e.g. infection due to Escherichia coli, Staphylococcus aureus, Klebsiella spp., and Streptococcus spp.) of one or more sows at specific stages of life, either qualitatively (e.g. in bins high/low, quartiles, above/below median etc.) or quantitatively.

Sow anti-microbial growth promoter model: This model has been trained and validated for use in predicting the use and/or benefit of specific anti-microbial growth promoters for one or more sows at specific stages of life, either qualitatively (e.g. in bins high/low, quartiles, above/below median etc.) or quantitatively.

Sow biotic health and growth promoter model: This model has been trained and validated for use in predicting the use and/or benefit of biotic (e.g., prebiotics, probiotics, postbiotics, antibiotics, phytogenics) health and growth promoters for one or more sows at specific stages of life, either qualitatively (e.g. in bins high/low, quartiles, above/below median etc.) or quantitatively.

Sow microbiome composition model: This model has been trained and validated for use in predicting the composition of the gut microbiome and associated metrics (such as richness and diversity) of one or more sows at specific stages of life, either qualitatively (e.g. in bins high/low, quartiles, above/below median etc.) or quantitatively.

Sow microbiome functional model: This model has been trained and validated for use in predicting the functional potential and/or capacity of the gut microbiome (as measured via the carbohydrate active enzyme activity, KEGG pathways, amongst others) of one or more sows at specific stages of life, either qualitatively (e.g. in bins high/low, quartiles, above/below median etc.) or quantitatively.

Sow microbiome network interaction model: This model has been trained and validated for use in predicting the interactions between the microbial members, including but not limited to hub species, and positive and negative interactions, in the gut of the one or more sows at specific stages of life, either qualitatively (e.g. in bins high/low, quartiles, above/below median etc.) or quantitatively.

Sow microbiome stability model: This model has been trained and validated for use in predicting the stability (as defined by the day-to-day variation in microbiome) of the microbiome of one or more sows at specific stages of the life, either qualitatively (e.g. in bins high/low, quartiles, above/below median etc.) or quantitatively.

Sow microbiome robustness model: This model has been trained and validated for use in predicting the robustness (as defined by the capacity of microbiome to resist changes caused by stressors) of the microbiome of one or more sows at specific stages of the life, either qualitatively (e.g. in bins high/low, quartiles, above/below median etc.) or quantitatively.

Sow microbiome resilience model: This model has been trained and validated for use in predicting the resilience (as defined by the capacity and the time it takes for the microbiome to return to equilibrium upon the act of stressors) of the microbiome of one or more sows at specific stages of the life, either qualitatively (e.g. in bins high/low, quartiles, above/below median etc.) or quantitatively.

Sow reproductive performance model: This model has been trained and validated for use in predicting the reproductive health and performance (e.g. measures such as litter size, average birth weight of piglets, litter weight at birth, number of total born, number of alive born, weak number, number of still births, mummy count, average alive weight, number of weaned piglets, average weaning weight, weaning litter weight, litter weight gain, average daily gain, weight gain of piglets, farrowing rate, weaning-to-estrus interval, average feed intake during lactation, pigs weaned per sow per year, lifetime performance, livability) of one or more sows, either qualitatively (e.g. in bins high/low, quartiles, above/below median etc.) or quantitatively.

Sow to piglet model: This model has been trained and validated for use in predicting the offspring (e.g., piglets) health (e.g., diarrhea score, livability), performance (body weight, average daily gain, average daily feed intake, gain-to-feed ratio) and gut microbiome features using the sow metagenomic data (e.g., fecal, rectal, vaginal, milk), either qualitatively (e.g. in bins high/low, quartiles, above/below median etc.) or quantitatively.

### Gilt Models

The gilt models can include models for predicting future health measures or future performance measures in gilts using microbiome data obtained from a microbiome sample including one or more gilt fecal samples, gilt rectal samples, or gilt vaginal samples.

By way of non-limiting example, the following gilt models are contemplated for use in the systems and methods described herein for gilts of any age in their life stage:

Gilt mortality model: This model has been trained and validated for use in predicting the mortality (e.g., probability of death) of one or more gilts at specific stages of life, either qualitatively (e.g. in bins high/low, quartiles, above/below median etc.) or quantitatively.

Gilt livability model: This model has been trained and validated for use in predicting the livability (e.g., probability of survival) of one or more gilts at specific stages of life, either qualitatively (e.g. in bins high/low, quartiles, above/below median etc.) or quantitatively.

Gilt general health model: This model has been trained and validated for use in predicting the general health status of one or more gilts at specific stages of life qualitatively (e.g. in bins good/bad, high/low, quartiles) or quantitatively.

Gilt gastrointestinal pathogen risk model: This model has been trained and validated for use in predicting the probability of incidence and/or severity of singular and/or plural gastrointestinal pathogen infection (e.g. including, but not to be limited to, infection due to Escherichia coli, Salmonella enterica, Streptococcus suis, Clostridium perfringens type A and C, Lawsonia intracellularis, Brachyspira hyodysenteriae, Eimeria sp., Isospora suis. Campylobacter coli) of one or more gilts at specific stages of life, either qualitatively (e.g. in bins high/low, quartiles, above/below median etc.) or quantitatively.

Gilt respiratory pathogen risk model: This model has been trained and validated for use in predicting the probability of incidence and/or severity of singular and/or plural respiratory pathogen infection (e.g. including, but not to be limited to, infection due to Streptococcus suis, Mycoplasma hyopneumoniae, Actinobacillus pleuropneumoniae, Glaesserella parasuis (Haemophilus parasuis), Pasteurella multocida, Bordetella bronchiseptica, Mycoplasma hyorhinis, porcine reproductive and respiratory syndrome virus (i.e., PRRS), influenza, porcine circovirus type 2) of one or more gilts at specific stages of life, either qualitatively (e.g. in bins high/low, quartiles, above/below median etc.) or quantitatively.

Gilt reproductive system pathogen risk model: This model has been trained and validated for use in predicting the probability of incidence and/or severity and/or from carrier/exposure risk perspective of singular and/or plural respiratory pathogen infection (e.g. including, but not to be limited to, infection due to porcine reproductive and respiratory syndrome virus (i.e., PRRS), porcine circovirus type 2/3) of one or more gilts at specific stages of life, either qualitatively (e.g. in bins high/low, quartiles, above/below median etc.) or quantitatively.

Gilt anti-microbial growth promoter model: This model has been trained and validated for use in predicting the use and/or benefit of specific anti-microbial growth promoters for one or more gilts at specific stages of life, either qualitatively (e.g. in bins high/low, quartiles, above/below median etc.) or quantitatively.

Gilt biotic health and growth promoter model: This model has been trained and validated for use in predicting the use and/or benefit of biotic (e.g., prebiotics, probiotics, postbiotics, antibiotics, phytogenics) health and growth promoters for one or more gilts at specific stages of life, either qualitatively (e.g. in bins high/low, quartiles, above/below median etc.) or quantitatively.

Gilt microbiome composition model: This model has been trained and validated for use in predicting the composition of the gut microbiome and associated metrics (such as richness, diversity) of one or more gilts at specific stages of life, either qualitatively (e.g. in bins high/low, quartiles, above/below median etc.) or quantitatively.

Gilt microbiome functional model: This model has been trained and validated for use in predicting the functional potential and/or capacity of the gut microbiome (as measured via the carbohydrate active enzyme activity, KEGG pathways, amongst others) of one or more gilts at specific stages of life, either qualitatively (e.g. in bins high/low, quartiles, above/below median etc.) or quantitatively.

Gilt microbiome network interaction model: This model has been trained and validated for use in predicting the interactions between the microbial members, including but not limited to hub species, and positive and negative interactions, in the gut of the one or more gilts at specific stages of life, either qualitatively (e.g. in bins high/low, quartiles, above/below median etc.) or quantitatively.

Gilt microbiome stability model: This model has been trained and validated for use in predicting the stability (as defined by the day-to-day variation in microbiome) of the microbiome of one or more gilts at specific stages of the life, either qualitatively (e.g. in bins high/low, quartiles, above/below median etc.) or quantitatively.

Gilt microbiome robustness model: This model has been trained and validated for use in predicting the robustness (as defined by the capacity of microbiome to resist changes caused by stressors) of the microbiome of one or more gilts at specific stages of the life, either qualitatively (e.g. in bins high/low, quartiles, above/below median etc.) or quantitatively.

Gilt microbiome resilience model: This model has been trained and validated for use in predicting the resilience (as defined by the capacity and the time it takes for the microbiome to return to equilibrium upon the act of stressors) of the microbiome of one or more gilts at specific stages of the life, either qualitatively (e.g. in bins high/low, quartiles, above/below median etc.) or quantitatively.

Gilt future reproductive performance model: This model has been trained and validated for use in predicting the reproductive health and performance (e.g. measures such as litter size, average birth weight of piglets, litter weight at birth, number of total born, number of alive born, weak number, number of still births, mummy count, average alive weight, number of weaned piglets, average weaning weight, weaning litter weight, litter weight gain, average daily gain, weight gain of piglets, farrowing rate, weaning-to-estrus interval, average feed intake during lactation, pigs weaned per sow per year, lifetime performance, livability, or age at first mating) of one or more gilts, either qualitatively (e.g. in bins high/low, quartiles, above/below median etc.) or quantitatively.

As used herein, the term “stability” as it relates to the microbiome refers to the day-to-day variation in the microbiome, such as the variation before and after a feeding, or the variation due to feed type or source.

As used herein, the term “robustness” as it relates to the microbiome refers to the capacity of the microbiome to resist changes that are imposed upon it by various stressors, such as pathogenic stressors or metabolic changes in the animal.

As used herein, the term “resilience” as it relates to the microbiome refers to the capacity and the time it takes for the microbiome to return to equilibrium or close to equilibrium after it has been pushed out of equilibrium by various stressors, such as recovery after a pathogenic stressor or a metabolic changes in the animal.

As used herein, the term “richness” as it relates to the microbiome refers to the number of taxa or functions in a given biological sample, such as a fecal sample, a rectal sample, a vaginal sample, and a mammary sample. Richness is a measure of the number of different types of taxa or functions within a given sample, where the types of taxa can be similar or different among samples.

As used herein, the term “diversity” as it relates to the microbiome refers to the richness and evenness of distribution of each taxon or functions in a given sample.

### Predictions, Recommendations, Interventions

The systems and methods herein can generate, and report customized predictions, recommendations, and interventions based on the metagenomics data, metadata, and target query and model executions.

Predictions can include those predictions regarding the future health measures or future performance measures of an animal. The predictions can include any predictions about a future status of one or more future health measures or future performance measures, including, but not to be limited to one or more of future body weight, future feed conversion ratio, future feed intake, future body composition, future meat quality (e.g., marbling, color, water holding capacity, and pH), future carcass quality, future growth rate, future average daily weight gain, future heart girth diameter, future livability, future mortality, future morbidity, future gastrointestinal pathogen risk, future respiratory pathogen risk, future reproductive system pathogen risk, future mammary glands pathogen risk, future litter size, future number of liveborn piglets, future number of stillborn piglets, future parity, future sow mortality, or future incidence of uterine prolapse, or any combination thereof, for any of a piglet, a sow or a gilt where developmentally appropriate.

Recommendations can include those recommendations regarding the future health measures or future performance measures of an animal. The recommendations can include, but are not to be limited to, a recommendation to change a feed or supplement composition, add or remove a feed or supplement composition, administer one or more vaccines, administer one or more medications, make alterations to a rearing environment or management system, select an animal including an indication for enhanced future health measures or future performance measures, culling an animal including an indication for diminished future health measures or future performance measures, select a gilt including an indication for enhanced future health measures or future performance measures, or add or remove anti-microbial growth promoters into a diet of the animal, or any combination thereof. The recommendations can include recommending dosing, duration, and timing of the administration one or more postbiotics, prebiotics, phytogenics, essential oils, or other nutrition to an animal's feed or animal's supplement. The recommendations can include adding one or more postbiotics, prebiotics, phytogenics, essential oils, or other nutrition to an animal's feed or animal's supplement. The recommendations can include removing one or more postbiotics, prebiotics, phytogenics, essential oils, or other nutrition to an animal's feed or animal's supplement. Recommendations can include selecting an animal as a high performer, high health, low performer that requires intervention, low health that requires intervention, etc. It will be appreciated that the recommendations can be provided to a user as customized management recommendations tailored to the needs of a specific user.

Interventions can include those interventions regarding the future health measures or future performance measures of an animal. The interventions can include, but are not to be limited to, one or more interventions including: changing a feed or supplement composition, supplying a feed or supplement composition, adding or removing a feed or supplement composition, administering one or more vaccines, administering one or more medications, making alterations to the rearing environment and management system, making alterations to the management system, selecting an animal including an indication for enhanced future health measures or future performance measures, culling an animal including an indication for diminished future health measures or future performance measures, selecting a gilt including an indication for enhanced future health measures or future performance measures, or adding or removing one or more anti-microbial growth promoters into a diet of the animal. It will be appreciated that the interventions can be provided to a user as customized management interventions including customized feed or supplement interventions tailored to the needs of a specific user. In an aspect, the interventions can be administered to an animal or animals to improve the future health and future performance of the animal. By way of non-limiting example, the interventions can include administering one or more feed compositions, supplement compositions, and the like, where the feed compositions and supplement compositions can include one or more of a microbial fermentate product, including a postbiotic fermentate product isolated from one or more strains of bacteria or yeast. The interventions further can include the addition of one or more phytogenics or essential oils. The interventions can include providing customized dosing, duration, and timing of the administration one or more postbiotics, prebiotics, phytogenics, essential oils, or other nutrition to an animal's feed or animal's supplement in order to optimize future health and performance of the animal.

### Process Flow

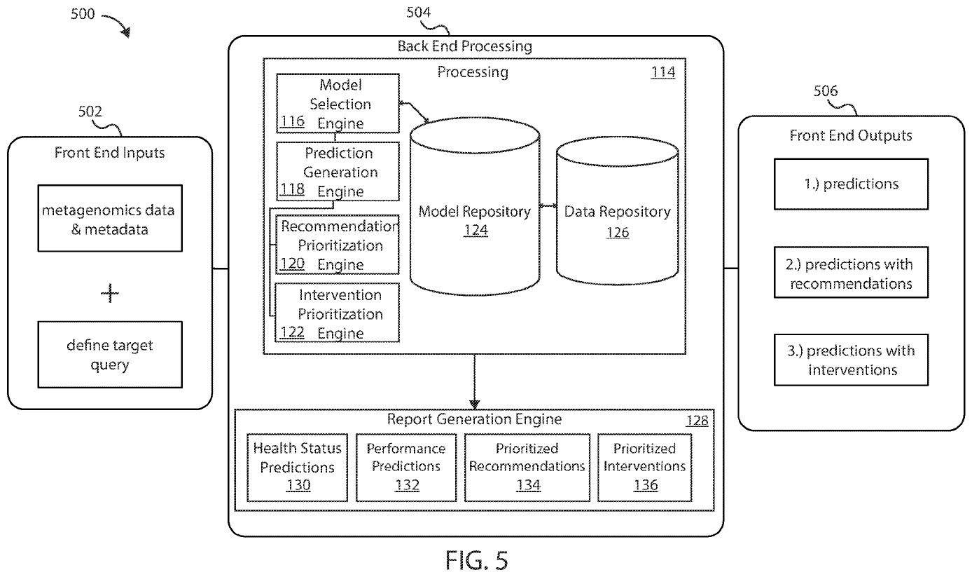

The process flow implemented by the multigenerational microbiome systems and methods herein can include a data input on a front end of the multigenerational microbiome system 100. Referring now to FIG. 5, an exemplary process flow 500 is provided, where the data input received on a front end inputs 502 includes an input of metadata, an input of microbiome data, and/or an input of a defined target query. The front end of the mutigenerational microbiome system can include a graphical user interface suitable for display of one or more user interfaces in a computer readable data structure provided through a web site, through a mobile application, and the like. The data input on the front end of the multigenerational microbiome system can be analyzed by the processing component 114 on a back end 504 of the multigenerational microbiome system, whereby model selection engine 116 is configured to select one or more models and the prediction generation engine 118 is configured to execute the one or more models as described in reference to FIG. 1. The multigenerational microbiome system can further generate one or more reports with the report generation engine 128 and provide the report to the front end outputs 506 in a computer readable data structure, such as a graphical user interface suitable for display provided through a web site, through a mobile application, and the like, to provide the user with one or more predictions, recommendations, and interventions.

### Methods

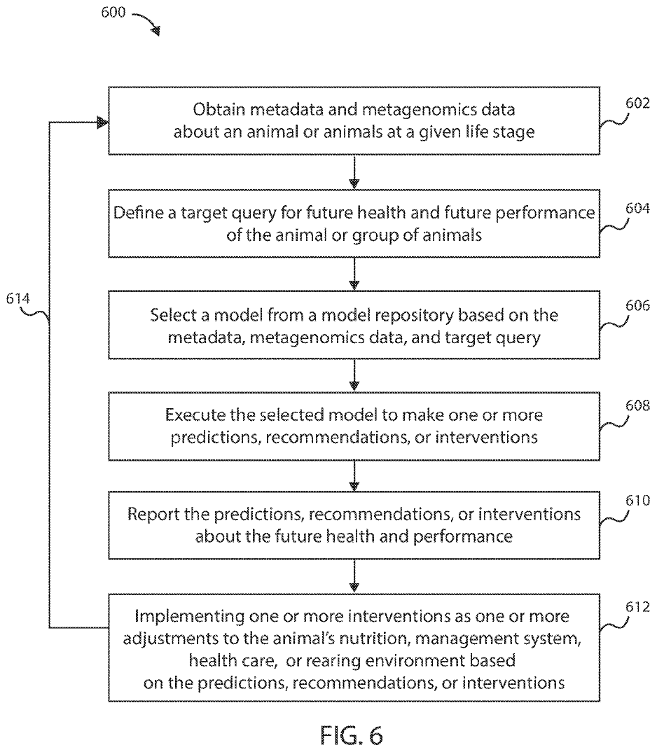

Various methods are provided that are implemented by the multigenerational microbiome systems herein. Referring now to FIG. 6, an exemplary method 600 is provided in accordance with the aspects herein. Method 600 can include a method for optimizing future health or future performance in an animal including obtaining a sample dataset indicative of an entire microbial community within a gastrointestinal tract, mammary glands, reproductive system, or respiratory system of the animal at 602. The method 600 further can include defining a query including one or more target queries for future health or future performance of the animal at 604. The method 600 further can include selecting a model from a model repository based on the sample dataset and the query including the one or more target queries at 606. The method 600 further can include executing the selected model using the sample dataset and the query including the one or more target queries to make predictions, generate recommendations, or generate interventions about future health or future performance of the animal at 608. The method 600 further can include reporting the predictions, recommendations, or interventions about future health or future performance, or combinations thereof at 610. The method 600 further can include implementing one or more interventions as one or more adjustments to the animal's nutrition, management system, health care, or rearing environment based on the predictions, recommendations, or interventions at 612. In an aspect, the method 600 can include obtaining a sample dataset indicative of an entire microbial community within a gastrointestinal tract of the animal. In an aspect, the method 600 can include obtaining a sample dataset indicative of an entire microbial community within the mammary glands of the animal. In an aspect, the method 600 can include obtaining a sample dataset indicative of an entire microbial community within a reproductive system of the animal. In an aspect, the method 600 can include obtaining a sample dataset indicative of an entire microbial community within a respiratory system of the animal.

In various aspects of the methods herein, the method 600 can be repeated at 614 as often as desired or required during an animal's lifecycle. By way of example, the method 600 can be repeated to monitor progress of the animal through life stage transitions, in response to an intervention, or in response to pathogen exposure. The method 600 can be repeated as many times as desired or required to update a feed or feed supplement, to administer a medication or vaccine, to make change to farm management system, or to alter rearing conditions.

In various aspects of the methods herein, each model selected from the model repository is generated by a model generation engine, where the model generation engine has been trained using one or more of metagenomics data or metadata obtained from one or more observational and interventional studies conducted on one or more past generations of animals. In an aspect, each model selected from the model repository can be periodically retrained using newly acquired data obtained from one or more metagenomics data or metadata obtained from one or more observational and interventional studies, or from one or more metagenomics data or metadata obtained from one or more production farm locations.

The sample data set suitable for use in the methods herein can include metagenomics data. In an aspect, the sample data set suitable for use in the methods herein can include metagenomics data and metadata, each of which are described elsewhere herein.

The sample data set suitable for use in the methods herein can include metagenomics data sourced from at least one microbiome sample including a fecal sample, a rectal sample, a vaginal sample, a nasal sample, an oral sample, a lung sample, or a mammary gland sample.

The targets for future health or future performance include one or more of body weight, feed conversion ratio, feed intake, growth rate, average daily weight gain, heart girth diameter, mortality, morbidity, gastrointestinal pathogen risk, respiratory pathogen risk, reproductive system pathogen risk, mammary glands pathogen risk, litter size, number of liveborn piglets, number of stillborn piglets, parity, sow mortality at farrowing, or incidence of uterine prolapse.

The methods here can identify a model from the model repository including one or more trained models such as a prediction model, a recommendation model, or an intervention model. The methods herein can identify a prediction model from the model repository, where the prediction model can include one or more predictions regarding the future performance or the future health of the animal. The one or more predictions can include a prediction of: future body weight, future feed conversion ratio, future feed intake, future growth rate, future average daily weight gain, future heart girth diameter, future livability, future mortality, future morbidity, future gastrointestinal pathogen risk, future respiratory pathogen risk, future reproductive system pathogen risk, future mammary glands pathogen risk, future litter size, future number of liveborn piglets, future number of stillborn piglets, future parity, future sow mortality, or future incidence of uterine prolapse, or any combination thereof.

The methods herein can identify a recommendation model from the model repository, where the recommendation model can include one or more recommendations regarding the future performance or the future health of the animal. The one or more recommendations can include a recommendation to: change a feed or supplement composition, add or remove a feed or supplement composition, administer one or more vaccines, administer one or more medications, make alterations to a rearing environment or management system, select an animal including an indication for enhanced future performance or future health, select a gilt including an indication for enhanced future performance or future health, or add or remove anti-microbial growth promoters into a diet of the animal, or any combination thereof.

The methods herein can identify an intervention model from the model repository where the intervention model can include one or more interventions regarding the future performance or the future health of the animal. The one or more interventions can include: changing a feed or supplement composition, supplying a feed or supplement composition, adding or removing a feed or supplement composition, administering one or more vaccines, administering one or more medications, making alterations to the rearing environment and management system, making alterations to the management system, selecting an animal including an indication for enhanced future performance or future health, selecting a gilt including an indication for enhanced future performance or future health, or adding or removing one or more anti-microbial growth promoters into a diet of the animal, or any combination thereof.

The predictions, recommendations, or interventions created by the methods herein are configured to reduce the incidence or severity of disease, improve health and performance measures in the animal, reduce the number of animals needing to be culled from a population, select individual animals including an indication for enhanced future performance or future health, identify individual animals that require interventions, or reduce the reliance on antimicrobial medications.

In an aspect, the methods herein are configured to select a model that has been generated based on metagenomics data from one or more past generations of animals.

In an aspect, the methods herein are configured to select a model that has been generated based on the type of metagenomics data and the body site where the sampled data was obtained.

In an aspect, the methods herein are configured to select one or more of a piglet model, a sow model, or a gilt model.

In an aspect, the methods herein are configured to select a piglet model that includes one or more of: a piglet growth rate model, a piglet average daily gain model, a piglet alpha diversity model, a piglet mortality model, a piglet livability model, a piglet general health model, a piglet gastrointestinal pathogen risk model, a piglet respiratory pathogen risk model, a piglet anti-microbial growth promoter model, a piglet biotic health and growth promoter model, a piglet microbiome composition model, a piglet microbiome functional model, a piglet microbiome network interaction model, a piglet microbiome stability model, a piglet microbiome robustness model, and a piglet microbiome resilience model.

In an aspect, the methods herein are configured to select a sow model that includes one or more of: a sow mortality model, a sow livability model, a sow general health model, a sow gastrointestinal pathogen risk model, a sow respiratory pathogen risk model, a sow reproductive system risk model, a sow mammary glands pathogen risk model, a sow anti-microbial growth promoter risk model, a sow biotic health and growth promoter model, a sow microbiome composition model, a sow microbiome functional model, a sow microbiome network interaction model, a sow microbiome stability model, a sow microbiome robustness model, a sow microbiome resilience model, a sow reproductive performance model, and a sow to piglet performance model.

In an aspect, the methods herein are configured to select a gilt model that includes one or more of: a gilt mortality model, a gilt livability model, a gilt general health model, a gilt gastrointestinal pathogen risk model, a gilt respiratory pathogen risk model, a gilt reproductive system risk model, a gilt anti-microbial growth promoter risk model, a gilt biotic health and growth promoter model, a gilt microbiome composition model, a gilt microbiome functional model, a gilt microbiome network interaction model, a gilt microbiome stability model, a gilt microbiome robustness model, a gilt microbiome resilience model, and a gilt future reproductive performance model.

In an aspect, the methods herein can include where the animal includes a swine animal, where the animal can include a swine animal as a piglet, a gilt, or a sow. In an aspect, the methods include obtaining a sample from just one animal. In another aspect, the methods include obtaining a sample from more than one animal.

In an aspect, the present disclosure provides a method for optimizing future health or future performance in an animal, where the method includes obtaining a sample dataset indicative of an entire microbial community within a reproductive system of the animal. The method further can include defining a query including one or more target queries for future health or future performance of the animal. The method further can include selecting a model from a model repository based on the sample dataset and the query comprising the one or more targets. The method further can include executing the selected model using the sample dataset and the query comprising the one or more targets to make predictions, generate recommendations, or generate interventions about future health or future performance of the animal. The method further can include reporting the predictions, recommendations, or interventions about future health or future performance, or combinations thereof. The method further can include implementing one or more interventions by providing a feed composition or supplement composition based on the predictions, recommendations, or interventions.

The methods herein can include where implementing the one or more interventions as one or more adjustments includes: switching from conventional rearing to an environment where the one or more animals are raised without antimicrobials; switching from an environment where the one or more animals are raised without antimicrobials to a conventional rearing with antimicrobials; switching to a system of rearing where the one or more animals are raised using alternatives to antimicrobial compounds comprising prebiotics, probiotics, postbiotics, or phytogenics; increasing or decreasing herd size; implementing one or more culling decisions; isolating one or more animals: adding or removing one or more supplement compositions; changing an existing supplement composition; adding or removing a supplement containing one or more anti-microbial growth promoters; adding or removing a feed composition or compositions to address one or more nutrition deficiencies; changing a feed composition; adding of one or more vitamins or minerals; adding or removing one or more anti-microbial growth promoters into a feed; administering one or more vaccines to prevent or treat a disease; or administering one or more medications to prevent or treat a disease.

The methods herein can include a method for predicting future health or future performance in an animal. The method can include obtaining a sample dataset indicative of an entire microbial community within a gastrointestinal tract, mammary gland, or reproductive system, or respiratory system of the animal. The method further can include defining a query including one or more target queries for future health or future performance of the animal. The method further can include selecting a model from a model repository based on the sample dataset and the query including the one or more targets, where each model selected from the model repository is generated by a model generation engine, and wherein the model generation engine has been trained using one or more of metagenomics data or metadata obtained from one or more observational and interventional studies. The method further can include executing the selected model using the sample dataset and the query including the one or more targets to make predictions, generate recommendations, or generate interventions about future health or future performance of the animal. The method further can include reporting the predictions, recommendations, or interventions about future health or future performance, or combinations thereof. The method further can include implementing one or more interventions as one or more adjustments to the animal's nutrition, management system, health care, or rearing environment based on the predictions, recommendations, or interventions.

The methods herein can include a method for optimizing future health or future performance in an animal. The method can include obtaining a sample dataset indicative of an entire microbial community within a reproductive system of the animal. The method further can include defining a query including one or more target queries for future health or future performance of the animal. The method further can include selecting a model from a model repository based on the sample dataset and the query including the one or more targets. The method further can include executing the selected model using the sample dataset and the query including the one or more targets to make predictions, generate recommendations, or generate interventions about future health or future performance of the animal. The method further can include reporting the predictions, recommendations, or interventions about future health or future performance, or combinations thereof. The method further can include implementing one or more interventions as one or more adjustments to the animal's nutrition, management system, health care, or rearing environment based on the predictions, recommendations, or interventions.

The methods herein can include a method for optimizing future health or future performance in an animal. The method can include obtaining a sample dataset indicative of an entire microbial community within a mammary gland of the animal. The method further can include defining a query including one or more target queries for future health or future performance of the animal. The method further can include selecting a model from a model repository based on the sample dataset and the query including the one or more targets. The method further can include executing the selected model using the sample dataset and the query including the one or more targets to make predictions, generate recommendations, or generate interventions about future health or future performance of the animal. The method further can include reporting the predictions, recommendations, or interventions about future health or future performance, or combinations thereof. The method further can include implementing one or more interventions as one or more adjustments to the animal's nutrition, management system, health care, or rearing environment based on the predictions, recommendations, or interventions.

The present disclosure provides a method for optimizing future health or future performance in an animal. The method can include obtaining a sample dataset indicative of an entire microbial community within a respiratory system of the animal. The method further can include defining a query including one or more target queries for future health or future performance of the animal. The method further can include selecting a model from a model repository based on the sample dataset and the query including the one or more targets. The method further can include executing the selected model using the sample dataset and the query including the one or more targets to make predictions, generate recommendations, or generate interventions about future health or future performance of the animal. The method further can include reporting the predictions, recommendations, or interventions about future health or future performance, or combinations thereof. The method further can include implementing one or more interventions as one or more adjustments to the animal's nutrition, management system, health care, or rearing environment based on the predictions, recommendations, or interventions.

### Health and Performance Measures

The methods and systems herein can be used to make predictions, recommendations, or interventions regarding the future health or future performance for any of a piglet, a sow, or a gilt. The future health or future performance can include one or more future health measures and future performance measures including body weight; birth weight; body composition; growth rate; average daily weight gain; feed conversion ratio; feed intake; mortality; morbidity; livability; heart girth diameter, including animal heart girth measurement; general health; susceptibility to disease, including pathogen risk (e.g., gastrointestinal pathogen risk, respiratory pathogen risk, reproductive system pathogen risk, or mammary gland pathogen risk); or reproductive system measures, including litter size, number of liveborn piglets, number of stillborn piglets, parity, sow mortality at farrowing, and uterine prolapse. As used herein, the term “general health” can refer to the day-to-day health status of an animal, including the absence of disease and the probability for developing or not developing disease in the future.

In an aspect, the general health of an animal or group of animals can be impacted by the function of the animal's gut health and immune health. Thus, future health measures further can include future gut health or future immune health function. As used herein, the term “gut health” can refer to the efficient and effective digestion of food by the digestive system (e.g., esophagus, stomach, gall bladder, liver, pancreas, spleen, small intestine (e.g., duodenum, jejunum, ileum), and large intestine (e.g., cecum, colon, rectum); the healthy balance of digestive tract physical environment, such as pH, osmolality, absence of excess gas, and a deficiency or excess of volatile fatty acids; the healthy characteristics of epithelia, such as the absence of increased intestinal permeability, reduction of epithelial integrity, and mucosal inflammation: the absence of abdominal pain as caused by one or more adverse health conditions; and the healthy balance of lumen or mucosa-associated microbiomes: or any combinations thereof. As used herein, the term “immune health” can refer to the standard functioning of the immune system as it is understood, where the immune system includes at least the mucous membranes of the nose, mouth, and throat; the tonsils; the lymph nodes; the thymus; the spleen; the large and small intestines; the bone marrow; the immune cells of the blood, including at least monocytes, lymphocytes, neutrophils, eosinophils, basophils, macrophages, erythrocytes, platelets, stem cells, and the like; and the skin.

### Animals

The systems and methods herein can be applied to any type of animal. In various aspects the animal can include any type of swine animal.

Swine (Sus domesticus) suitable for analysis using the multigenerational microbiome systems and methods herein can include, but are not limited to, breeds including American Landrace, American Yorkshire, Angeln Saddleback, Ba Xuyen, Berkshire, Bentheim Black Pied, Black Iberian, British, Landrace, British Saddleback, Chester White, Choctaw, Cinta Senese, Danish Landrace, Danish Protest, Duroc, Dutch Landrace, Gascon, Gloucestershire Old Spots, Guinea, Hampshire, Hereford, Large Black, Large White, Limousin, Lincolnshire Curly, Jeju Black, Juliana, Kagoshima Berkshire, Kunekune, Mangalica, Meishan, Middle White, Micro, Moura, Mulefoot, Nustrale, Pietrain, Poland China, Ossabaw Island, Oxford Sandy and Black, Sarda, Small White, Red Wattle, Swabian-Hall. Tamworth. Thuoc Nhieu, Tokyo-X. Vietnamese Pot-bellied.

The swine can include, but are not to be limited to, piglets, gilts, sows, baconers, barrows, boars, dams, feeders, growers, pigs, porkers, runts, sires, stags, or hogs. The animals herein can be analyzed at any number of developmental life stages. The life stages can include any of birth, pre-weaning, weaning, post-weaning, pre-farrowing, farrowing, post-farrowing, gestating, lactating, and postpartum. In various aspects, the animals suitable for the systems and methods described herein can include piglets, gilts, or sows. In an aspect, the animal is a piglet. In an aspect, the animal is a gilt. In an aspect, the animal is a sow.

### Computing Environment

The multigenerational microbiome system can include a computer system and components associated therewith. Referring now to FIG. 7 a schematic representation of a computer system 700 is provided in accordance with the various aspects of the systems and methods described herein. The computer system 700 is exemplary of one or more of the computing resources discussed herein.

In various aspects, the computer system 700 can operate as a standalone device or can be connected (e.g., via a network) to other computers or computing components. In a networked deployment, the computer system 700 can operate in the capacity of either a server or a client machine in server-client network environments, or it can act as a peer machine in peer-to-peer (or distributed) network environments. The computer system 700 can include a personal computer (PC), a tablet PC, a hybrid tablet, a personal digital assistant (PDA), a mobile telephone, or any machine capable of executing instructions (sequential or otherwise) that specify actions to be taken by that machine. Further, while only a single computer system 700 is illustrated, the term “computer” shall also be taken to include any collection of computers that individually or jointly execute a set (or multiple sets) of instructions to perform any one or more of the methods discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Exemplary computer system 700 includes at least one processor 702 (e.g., a central processing unit (CPU), a graphics processing unit (GPU) or both, processor cores, compute nodes, etc.), a main memory 704 and a static memory 706, which communicate with each other via a link 708 (e.g., bus). The computer system 700 can further include a video display unit 710, an alphanumeric input device 712 (e.g., a keyboard), and a user interface (UI) navigation device 714 (e.g., a mouse or trackpad). In one example, the video display unit 710, input device 712 and UI navigation device 714 are incorporated into a touch screen display. The computer system 700 can additionally include a storage device 716 (e.g., a drive unit), such as a global positioning system (GPS) sensor, compass, accelerometer, gyroscope, magnetometer, or other sensors.

The storage device 716 includes a machine-readable medium 720 on which is stored one or more sets of data structures and instructions 722 (e.g., software) embodying or utilized by any one or more of the methodologies or functions described herein. The instructions 722 can also reside, completely or at least partially, within the main memory 704, static memory 706, and/or within the processor 702 during execution thereof by the computer system 700, with the main memory 704, static memory 706, and the processor 702 also constituting machine-readable media.

While the machine-readable medium 720 is illustrated in an example to be a single medium, the term “machine-readable medium” can include a single medium or multiple media (e.g., a centralized or distributed database, and/or associated caches and servers) that store the one or more instructions 722. The term “machine-readable medium” shall also be taken to include any tangible medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 722 can further be transmitted or received over a communications network 724 using a transmission medium via the network interface device 718 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Bluetooth, Wi-Fi, 3G, and 4G LTE/LTE-A, 5G, DSRC, or WiMAX networks). The term “transmission medium” shall be taken to include any intangible medium that is capable of storing, encoding, or carrying instructions for execution by the machine, and includes digital or analog communications signals or other intangible medium to facilitate communication of such software.

Aspects herein can be implemented in one or a combination of hardware, firmware, and software. Aspects herein can also be implemented as instructions stored on a machine-readable storage device, which can be read and executed by at least one processor to perform the operations described herein. A machine-readable storage device can include any non-transitory mechanism for storing information in a form readable by a machine (e.g., a computer). For example, a machine-readable storage device can include read-only memory (ROM), random-access memory (RAM), magnetic disk storage media, optical storage media, flash-memory devices, and other storage devices and media.

A processor subsystem can be used to execute the instruction on the machine-readable medium. The processor subsystem can include one or more processors, each with one or more cores. Additionally, the processor subsystem can be disposed on one or more physical devices. The processor subsystem can include one or more specialized processors, such as a graphics processing unit (GPU), a digital signal processor (DSP), a field programmable gate array (FPGA), or a fixed function processor.

Examples, as described herein, can include, or can operate on, logic or a number of components, modules, or mechanisms. Modules can be hardware, software, or firmware communicatively coupled to one or more processors in order to carry out the operations described herein. Modules can be hardware modules, and as such modules can be considered tangible entities capable of performing specified operations and can be configured or arranged in a certain manner. In an example, circuits can be arranged (e.g., internally or with respect to external entities such as other circuits) in a specified manner as a module. In an example, the whole or part of one or more computer systems (e.g., a standalone, client or server computer system) or one or more hardware processors can be configured by firmware or software (e.g., instructions, an application portion, or an application) as a module that operates to perform specified operations. In an example, the software can reside on a machine-readable medium. In an example, the software, when executed by the underlying hardware of the module, causes the hardware to perform the specified operations. Accordingly, the term hardware module is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules includes a general-purpose hardware processor configured using software; the general-purpose hardware processor can be configured as respective different modules at different times. Software can accordingly configure a hardware processor, for example, to constitute a particular module at one instance of time and to constitute a different module at a different instance of time. Modules can also be software or firmware modules, which operate to perform the methods described herein.

Circuitry or circuits, as used in this document, can include, for example, singly or in any combination, hardwired circuitry, programmable circuitry such as computer processors including one or more individual instruction processing cores, state machine circuitry, and/or firmware that stores instructions executed by programmable circuitry. The circuits, circuitry, or modules can, collectively or individually, be embodied as circuitry that forms part of a larger system, for example, an integrated circuit (IC), system on-chip (SoC), desktop computers, laptop computers, tablet computers, servers, smart phones, etc.

As used in any example herein, the term “logic” can refer to firmware and/or circuitry configured to perform any of the aforementioned operations. Firmware can be embodied as code, instructions or sets of instructions and/or data that are hard-coded (e.g., nonvolatile) in memory devices and/or circuitry.

### EXAMPLES

Various aspects of the present disclosure can be better understood by reference to the following Examples, which are offered by way of illustration. The present disclosure is not limited to the Examples given herein.

### Example 1: Experimental Study Design

Examples 2-6 that follow are based on an investigation of the microbiome on future body weight measurements and future heart girth measurements in piglets. The animals included a total of 90 sows and a total of 360 piglets spread across nine different farms in two Canadian provinces. A total of seven visits to each of the farms were used to perform body weight measurements of all of the animals. Actual body weights were calculated for the piglets from the first to fourth visit, and additional body weights were calculated using the heart girth method from the fifth to the seventh visit. Heart girth measurements were obtained according to the methods of Groesbeck et al. (See: Groesbeck, et al., Using heart girth to determine weight in finishing pigs. Kansas State University, Agricultural Experiment Station and Cooperative Extension Service, (2002). pp. 166-168.) Visits are referred to throughout the Examples as Visits 1-7. Visit 1 was conducted at a median of 4 days±1 days, Visit 2 was conducted at a median 11 days±2 days, Visit 3 was conducted at a median of 18 days±2 days, Visit 4 was conducted at a median 27 days±3 days, Visit 5 was conducted at a median 60 days±7 days, Visit 6 was conducted at a median 97 days±15 days, and Visit 7 was conducted at a median 157 days±12 days. Microbiome data was collected from the piglet fecal samples from the first to the fourth visits, and from sow vaginal samples or sow fecal samples as indicated in the Examples. The fresh fecal samples for piglets or sows were obtained either by inserting a swab into fresh feces or by inserting a swab into the rectum of an animal. The sow vaginal samples were obtained by inserting a swab into the vagina of the sows.

### Example 2: Identification of Piglet Clusters Leading to Future Low and High Body Weight or Low and High Heart Girth

The analysis in this example investigated the differences in the performance of piglets at various time points of piglet production life (from birth to slaughter), investigating in particular pre-weaning and post-weaning effects on future body weight measurements or future heart girth measurements.

Briefly, pre-weaning microbiome samples were obtained from piglet fecal samples and were clustered separately to generate pre-weaning clusters (PRWC). Similarly, post-weaning microbiome samples were obtained from piglet fecal samples and were clustered separately to generate post-weaning clusters (POWC). In both cases no pruning of the genus was performed. Optimal number of cluster identification and clustering was performed using Gap Statistics in MATLAB, using the Spearman correlation for genus and Aitchison distance for samples. Clusters are considered as sub-groups of the samples based on similarity of the microbial composition.

The body weight measurements or heart girth measurements were binned at a particular time (at later ages between 66 days and 130 days of age) based on which cluster the piglet belonged to at the earlier time point of clustering. Statistical analyses were performed to check for statistical differences between the sub-groups using Wilcoxon Rank Sum Test and false discovery rate correction (using the BH, or Benjamini-Hochberg procedure). Clusters are defined as summarized in Table 1.

TABLE 1
Pre-Weaning and Post-Weaning Clusters

| Piglet Age (median) | Cluster(s) |
|---|---|
| 1 week post birth (4 days ± 1 day) at | PRWC 3; PRWC 4 |
| visit 1 | |
| 2 weeks post birth (11 days ± 2 days) at | PRWC 1; PRWC 2; PRWC 5; |
| visit 2 | PRWC 6 |
| 3 weeks post birth (18 days ± 2 days) at | PRWC 2; PRWC 5; PRWC 6 |
| visit 3 | |
| 4 weeks post birth (27 days ± 3 days) at | POWC 1; POWC 2; POWC3; |
| visit 4 | POWC 4; POWC 5; POWC 6 |

In the first week after birth (i.e., Visit 1 at median age 4 days±1 days), the samples primarily clustered into PRWC 3 and PRWC 4. As the piglets matured, by the second week of life (i.e., Visit 2 at median age 11 days±2 days), it was observed that the samples spread between PRWC 1, PRWC 2, PRWC 5 and PRWC 6. By three weeks after birth (i.e., Visit 3 at median age 18 days±2 days) and approaching weaning age, the samples clustered into PRWC 2, PRWC 5, and PRWC 6. During the first week post-weaning at four weeks of life (i.e., Visit 4 at median age 27 days±3 days), the clusters spread into six different POWC, including POWC 1, POWC 2, POWC3, POWC 4, POWC 5, and POWC 6.

Various predictions about future hearth girth measurements and body weight were made based on the clustering patterns. It was found that cluster assignment at week 1 of life was not indicative in terms of heart girth measurements at later time points in life. Piglets belonging to PRWC 2 in week 2 of life eventually displayed smaller heart girth measurements, which became more evident approaching 130 days of age. Piglets belonging to PRWC 6 in week 3 of life eventually had higher heart girth measurements, which became more evident with later age. Piglets belonging to POWC 1, POWC 2, and POWC 6 in week 4 of life, eventually had higher heart girth inch measurements, which became more evident with later age. Piglets belonging to POWC 3 and POWC 4 in week 4 of life, eventually had lower heart girth measurements, which became more evident with later age.

It was found that cluster assignment at week 1 of life was not indicative in terms of body weight measurements at later time points of piglet life. Piglets belonging to PRWC 2 in week 2 of life, eventually had smaller body weight measurements, which became more evident with later age. Piglets belonging to PRWC 6 in week 3 of life, eventually had higher body weight measurements, which became more evident with later age. Piglets belonging to POWC 1, POWC 2, and POWC 6 in week 4 of life, eventually had higher body weight measurements, which became more evident with later age. Piglets belonging to POWC 3 and POWC 4 in week 4 of life, eventually had lower body weight measurements, which became more evident with later age. Overall there are specific PRWC and POWC that are either more prone to have higher or lower heart girth measurements and body weight measurements at later ages.

The p-values of the statistical comparisons of the sub-groups for heart girth measurements at 97 days±15 days of life based on groupings in PRWC are reported in Table 2.

TABLE 2
Statistical Comparisons in PRWC Subgroups
for Heart Girth Measurements

| | Visit 1 | Visit 2 | Visit 3 |
|---|---|---|---|
| Comparisons | (p-values) | (p-values) | (p-values) |
| PRWC 1 versus PRWC 2 | NA | 0.0025 | 0.3964 |
| PRWC 1 versus PRWC 3 | NA | 1.0000 | 0.6122 |
| PRWC 1 versus PRWC 4 | NA | 1.0000 | NA |
| PRWC 1 versus PRWC 5 | NA | 1.0000 | 0.4580 |
| PRWC 1 versus PRWC 6 | NA | 1.0000 | 0.0260 |
| PRWC 2 versus PRWC 3 | 0.0656 | 0.2745 | 0.5232 |
| PRWC 2 versus PRWC 4 | 0.1905 | 0.2230 | NA |
| PRWC 2 versus PRWC 5 | 0.1905 | 0.0000 | 0.5861 |
| PRWC 2 versus PRWC 6 | 0.0816 | 0.0000 | 0.0000 |
| PRWC 3 versus PRWC 4 | 0.2497 | 1.0000 | NA |
| PRWC 3 versus PRWC 5 | 0.6414 | 1.0000 | 0.7284 |
| PRWC 3 versus PRWC 6 | 0.7399 | 1.0000 | 0.6006 |
| PRWC 4 versus PRWC 5 | 0.9572 | 1.0000 | NA |
| PRWC 4 versus PRWC 6 | 0.4938 | 1.0000 | NA |
| PRWC 5 versus PRWC 6 | 0.5000 | 1.0000 | 0.0022 |

The p-values of the statistical comparisons of the sub-groups for heart girth measurements at 97 days±15 days of life based on groupings in POWC are reported in Table 3.

TABLE 3
Statistical Comparisons in POWC Subgroups
for Heart Girth Measurements

| | Visit 4 |
|---|---|
| Comparisons | (p-values) |
| POWC 1 versus POWC 2 | 0.3927 |
| POWC 1 versus POWC 3 | 0.0012 |
| POWC 1 versus POWC 4 | 0.0029 |
| POWC 1 versus POWC 5 | 0.0215 |
| POWC 1 versus POWC 6 | 0.1268 |
| POWC 2 versus POWC 3 | 0.0080 |
| POWC 2 versus POWC 4 | 0.0257 |
| POWC 2 versus POWC 5 | 0.1097 |
| POWC 2 versus POWC 6 | 0.6999 |
| POWC 3 versus POWC 4 | 0.6306 |
| POWC 3 versus POWC 5 | 0.5477 |
| POWC 3 versus POWC 6 | 0.0033 |
| POWC 4 versus POWC 5 | 0.6306 |
| Cluster 4 versus Cluster 6 | 0.0090 |
| Cluster 5 versus Cluster 6 | 0.1097 |

### Example 3: Identification of Sow Clusters Leading to Low and High Body Weight Measurements or Low and High Heart Girth Measurements in Piglets

The analysis in this example investigated the differences in the sow microbiome on the future performance of piglets, investigating in particular the microbiome of sow fecal samples and sow vaginal samples on the body weight measurements or heart girth measurements in future generations of piglets.

Briefly, the sow fecal microbiome samples were clustered separately into sow fecal clusters (SFC) and sow vaginal microbiome samples were clustered separately into sow vaginal clusters (SVC). In both cases no pruning of the genus was performed. Optimal number of cluster identification and clustering was performed using Gap Statistics in MATLAB, using the Spearman correlation for genus and Aitchison distance for samples. Clusters are considered as sub-groups of the samples based on similarity of the microbial composition.

The body weight measurements or heart girth measurements were binned at a particular time (at visits between 66 days and 130 days of age) based on which cluster the piglet belonged to at the sow level. Statistical analyses were performed to check for statistical differences between the sub-groups using one or more generalized linear mixed models controlling for the effects of province, farm, and sow, and then adjusting all the P-values together. Sow fecal samples clustered into four optimal SFCs, including SFC 1, SFC 2, SFC 3, and SFC 4. Sow vaginal samples clustered into five optimal SVCs, including SVC 1, SVC 2, SVC 3, SVC 4, and SVC 5.

Results indicate that assignment to SFC 1 was indicative of lower heart girth measurements in piglets approaching 130 days of age. Assignment to SFC 2 was indicative of higher heart girth measurements in piglets approaching 130 days of age. Assignment to SVC 3 was indicative of lower heart girth measurements in piglets approaching 130 days of age. Assignment to any of SVC 1, SVC 2, and SVC 5 was indicative of higher heart girth measurements in piglets approaching 130 days of age. Overall, the data suggest that there are sow specific SRCs and SVCs that are prone to have higher or lower heart girth measurements and body weight measurements between 66 days and 130 days of age.

### Example 4: Determining a Correlation Between Microbe Type and Body Weight Measurements

The analysis in this example investigated which microbe types in piglet fecal samples are correlated with future body weight gain in piglets.

The methods used to identify which microbes are associated with body weight gain in piglets include a combination of techniques. These include analysis using Pearson correlation, Limma, and MaAsLin techniques. The Pearson correlation measures the statistical relationship, or association, between different microbes in the microbiome and body weight measures based on covariance. The Limma technique operates using an empirical Bayes method that estimates the prior from the set of all features. It can moderate the sample variances, which include mean squared deviations (e.g., a type of sample mean). The MaAsLin analysis relies on general linear models to accommodate most modern epidemiological study designs, including cross-sectional and longitudinal designs, and it offers a variety of data exploration, normalization, and transformation options.

The microbes highly positively correlated with body weight were assessed across all three techniques, and the results indicate that among the top 50 microbes identified there was an overlap of 42 microbes across all three techniques. In this example, 51 microbes were identified by MaAsLin to be highly positively correlated with body weight gain, 51 microbes were identified by Limma to be highly positively correlated with body weight gain, and 50 microbes were identified by Limma to be highly positively correlated with body weight gain. The microbes identified by each technique are summarized in Table 4.

TABLE 4
Microbes Positively Correlated with Body Weight

| Microbe | Pearson | Limma | MaAsLin |
|---|---|---|---|
| Prevotellaceae_NK3B31_group | 0.34709672 | 1.33631E−35 | 4.955E−15 |
| Treponema | 0.41566571 | 9.07938E−52 | 3.3769E−11 |
| Rikenellaceae RC9 gut group | 0.43952461 | 2.40204E−58 | 3.9589E−15 |
| Lachnospiraceae ND3007 group | 0.40738912 | 1.32743E−49 | 6.4899E−11 |
| Prevotella 7 | 0.40755482 | 1.19745E−49 | 7.8236E−19 |
| Phascolarctobacterium | 0.44415004 | 1.1105E−59 | 8.3836E−26 |
| Prevotellaceae UCG 001 | 0.34455 | 4.52E−35 | 1.09E−06 |
| Eubacterium hallii group | 0.377439 | 2.82E−42 | 2.66E−15 |
| Succinivibrio | 0.265786 | 5.08E−21 | 6.51E−21 |
| Alloprevotella | 0.401618 | 3.90E−48 | 3.81E−20 |
| Prevotellaceae_UCG_003 | 0.497992 | 7.26E−77 | 1.67E−24 |
| Intestinibacter | 0.401288 | 4.77E−48 | 8.94E−10 |
| Candidatus_{—}Soleaferrea | 0.359065 | 3.80E−38 | 2.17E−10 |
| Faecalibacterium | 0.520006 | 7.98E−85 | 2.56E−12 |
| Oscillospira | 0.466222 | 2.34E−66 | 2.66E−11 |
| Clostridium_{—}sensu_{—}stricto_6 | 0.36524 | 1.68E−39 | 5.73E−07 |
| Monoglobus | 0.373516 | 2.25E−41 | 8.17E−10 |
| Ruminococcus_{—}gauvreauii_group | 0.33903 | 6.08E−34 | 6.78E−16 |
| UCG_002 | 0.408092 | 8.68E−50 | 2.92E−10 |
| UCG_008 | 0.395608 | 1.25E−46 | 3.85E−10 |
| Lachnospiraceae XPB1014 group | 0.262239 | 1.74E−20 | 8.38E−07 |
| UCG 005 | 0.511606 | 1.02E−81 | 2.33E−20 |
| Eubacterium_{—}ruminantium_group | 0.309541 | 2.73E−28 | 9.66E−06 |
| Lachnospira | 0.317114 | 1.11E−29 | 2.27E−09 |
| Prevotella 9 | 0.575261 | 1.3527E−107 | 2.16E−37 |
| Coprococcus | 0.530452 | 8.28E−89 | 5.63E−14 |
| Anaerovibrio | 0.489857 | 4.55E−74 | 1.69E−16 |
| Lachnospiraceae UCG 010 | 0.385589 | 3.38E−44 | 3.05E−12 |
| Lachnospiraceae NK4A136 group | 0.386775 | 1.75E−44 | 3.34E−17 |
| Shuttleworthia | 0.350375 | 2.77E−36 | 2.28E−22 |
| Family_XIII_AD3011_group | 0.471067 | 6.88E−68 | 2.04E−15 |
| Oscillibacter | 0.449208 | 3.62E−61 | 2.95E−06 |
| Megasphaera | 0.295977 | 6.68E−26 | 2.61E−11 |
| UCG_009 | 0.365836 | 1.23E−39 | 1.30E−07 |
| Fusicatenibacter | 0.382635 | 1.71E−43 | 1.09E−21 |
| Incertae_{—}Sedis | 0.276186 | 1.24E−22 | 4.24E−11 |
| Agathobacter | 0.529617 | 1.74E−88 | 5.49E−33 |
| Christensenellaceae_R_7_group | 0.493267 | 3.12E−75 | 3.47E−12 |
| Fibrobacter | 0.283215 | 9.21E−24 | 3.42E−06 |
| Blautia | 0.387203 | 1.38E−44 | 3.11E−18 |
| NK4A214 group | 0.439146 | 3.08E−58 | 1.40E−09 |
| Subdoligranulum | 0.57083 | 1.32E−105 | 1.75E−14 |
| Lachnospiraceae UCG 010 | 0.385589 | 3.38E−44 | 3.05E−12 |

This example further investigated which microbe types are associated with body weight for piglets in groups identified as predicted to have very high future body weight and groups identified as predicted to have very low future body weight.

The weight gain of the piglets between visits 1 and 4 were calculated as follows: weight gain per day=(weight of the pig at a given visit−the initial weight of the pig at visit 1)/duration. The piglets were grouped on the basis of weight gain into five groups including: VL (Very Low), L (Low), M (Medium), H (High) and VH (Very High), with each group including approximately 230 samples per group (e.g., n=230 or n=232). Identification of microbes whose abundances were significantly different between the pigs in the VL and VH groups was conducted and the q-value (i.e., false discovery rate (BH) adjusted P-value) for each is summarized in Table 5 and Table 6.

TABLE 5
Microbes Positively Contributed and
Associated with BW in VH Group

| Microbe | q value |
|---|---|
| Intestinibacter | 2.02E−08 |
| Agathobacter | 1.23E−06 |
| Shuttleworthia | 1.23E−06 |
| Lachnospiraceae_UCG_003 | 1.70E−05 |
| Succinivibrio | 0.000367 |
| Lachnospiraceae_ND | 0.000558 |
| 3007_group | |
| Ruminococcus_{—}gauvreauii_{—} | 0.000677 |
| group | |
| Lachnospira | 0.000981 |
| Fusicatenibacter | 0.001082 |
| Prevotella_9 | 0.00185 |
| Aliivibrio | 0.005842 |
| UCG_003 | 0.028676 |
| Prevotellaceae_NK3B | 0.030389 |
| 31 group | |
| Amnipila | 0.030969 |
| Anaerovorax | 0.030969 |
| Monoglobus | 0.034515 |
| possible genus Sk018 | 0.041248 |
| Family_XIII_AD3011 | 0.044514 |
| group | |

TABLE 6
Microbes Positively Contributed and
Associated with BW in VL Group

| Microbe | q value |
|---|---|
| Prevotellaceae_UCG_001 | 1.29E−08 |
| Eubacterium_{—}xylanophilum_group | 1.29E−08 |
| Prevotella_9 | 2.32E−08 |
| Agathobacter | 3.36E−08 |
| Shuttleworthia | 1.82E−06 |
| Anaerovibrio | 2.36E−06 |
| Lachnospiraceae UCG 003 | 5.75E−06 |
| Succinivibrio | 7.27E−06 |
| UCG 009 | 1.18E−05 |
| Eubacterium siraeum group | 1.76E−05 |
| Ruminococcus gauvreauii group | 6.58E−05 |
| Family XIII AD3011 group | 8.14E−05 |
| Fusicatenibacter | 0.000189 |
| Lachnospiraceae_UCG_004 | 0.000453 |
| Alloprevotella | 0.000563 |
| Prevotellaceae_NK3B31_group | 0.000799 |
| UCG_005 | 0.000837 |
| Lachnospiraceae_ND3007_group | 0.001267 |
| Prevotellaceae_UCG_003 | 0.003244 |
| Amnipila | 0.004823 |
| Alistipes | 0.006407 |
| Prevotella_7 | 0.012315 |
| Unidentified | 0.019377 |
| Blautia | 0.021143 |
| Catenibacillus | 0.029267 |
| Incertae_{—}Sedis | 0.031655 |
| Phascolarctobacterium | 0.03357 |
| Streptococcus | 0.034308 |
| Oscillibacter | 0.036183 |
| possible genus Sk018 | 0.037677 |

In the VH group, 18 microbes were found to positively contribute to and be significantly associated with BW as tabulated in Table 5. In the VL group, 30 microbes were found to positively contribute to and be significantly associated with BW as tabulated in Table 6. Of these, 3 microbes were uniquely associated with the VH group and not in the VL group, including Intestinibacter, Coprococcus and Lachnospiraceae NK4A136 group.

### Example 5: Predicting Piglet Body Weight Measurements Using Linear Regression Models

The analysis in this example examined the predictability of the body weight of piglets using microbiome data and metadata with linear regression models.

Multiple linear regression (MLR) modeling was used to predict body weight of the piglets at visits 5 to 7 based on metadata and metagenomics data obtained during visits 1 to 4. The following combinations of data were used to create the models applied to predict future body weights: metadata only, metagenomics data only, and metadata combined with metagenomics data. The metagenomics data was sourced from fecal samples taken from piglets at approximately 3 days (visit 1), 10 days (visit 2), 18 days (visit 3), and 27 days (visit 4) of age. More than 30 models were generated using various combinations of metadata and microbiome data. The metadata included in the model development included one or more combinations of the following types of metadata (with query parameters in parentheses): body weight in kilograms (e.g., BW_kg), farm location (e.g., farm), litter cohort, (e.g., cohort), parity (e.g., parity), litter size (e.g., litter_size), live born piglets (e.g., live born), still birth piglets (e.g., stil_birth), sex (e.g., sex), visit number (e.g., visit), age (e.g., age), grouping including VL, L, M, H, and VH (e.g., grouping), selected model (e.g., all genus, 30P genus, 30N genus, etc.), and pig identification number (e.g., pig_id). The models that that included a combination of metadata and microbiome data had the highest R² values when compared to other models, as tabulated in Table 7.

TABLE 7
MLR models with different attributes and its performance.

| | | | | | Mean |
|---|---|---|---|---|---|
| | | | Root-mean- | | absolute |
| Model with Metadata | Multiple | Adjusted | square error | Mean | percentage |
| Parameters | R² | R² | (RMSE) | error | error |
| BW_kg~farm + cohort + | 0.8232 | 0.8204 | 1.096 | −0.013 | 19.94 |
| parity + litter_size + live_born + | | | | | |
| stil_birth + Sex + Visit + | | | | | |
| Age | | | | | |
| BW_kg~farm + parity + Sex + | 0.8978 | 0.896 | 0.833 | 0.049 | 16.539 |
| Age + Grouping | | | | | |
| BW_kg~farm + parity + Sex + | 0.8987 | 0.8987 | 0.828 | 0.034 | 16.397 |
| Visit + Age + Grouping | | | | | |
| BW_kg~farm + parity + Sex + | 0.9532 | 0.9113 | 2.879 | 0.132 | 35.919 |
| Visit + Age + Grouping + | | | | | |
| All Genus | | | | | |
| BW_kg~farm + parity + Sex + | 0.9098 | 0.9029 | 0.867 | 0.02 | 17.818 |
| Age + Grouping + 30P | | | | | |
| Genus + 30N Genus | | | | | |
| BW_kg~farm + parity + | 0.9122 | 0.9053 | 0.854 | 0.033 | 17.804 |
| litter_size + live_born + | | | | | |
| stil_birth + Age + Grouping + | | | | | |
| 30P Genus + V30N Genus | | | | | |

### Example 6: Predictive Models for Piglet Body Weight Gain, Piglet Litter Size, and Proportion of Live Piglet Births

The analysis in this example examined the various predictive models for determining future piglet body weight, future piglet litter size, and future proportion of live piglet births.

A set of classification models was developed using either the piglet microbiome data or the sow microbiome as inputs to predict future outcomes at the piglet and sow levels. Classification models were chosen as a starting point to evaluate performance metrics. Thus, the objective of the model development was to use the microbiome of individual animals to predict future performance (e.g., health measures, growth measures, other performance measures, and reproductive measures) to establish predictions for targeted interventions using feeds or feed supplements that can provide the animals with a tailored nutrition regime to catch up to their higher performing counterparts with and/or to improve their resistance to disease. Both piglet models and sow models were developed.

Piglet models: by leveraging the piglet fecal microbiome data, information was used to a.) determine an optimal sampling window (as the piglet microbiome was assessed at different points in time) to maximize the predictive power of piglet performance, which is as a surrogate of average daily gain used for performance; and b.) identify the optimal period (as growth can be measured over different periods of time) to target for prediction that maximizes performance, and is also practical and feasible for sampling.

Performance was measured by computing average daily weight gain (ADG) between different points in time. From visits 5 to 7, body weight was not measured, but instead was estimated based on the heart girth measurement at that time. As classification models require a categorical target, the estimated ADGs between different periods were dichotomized into high and low ADG around the observed median.

Various models were developed, including 1.) all pre-weaning piglet microbiome samples (i.e. visits 1, 2 and 3) used to predict ADG between visits 1 and 5, and between visits 1 and 7; and 2.) microbiome fecal samples collected from piglets at visits 1, 2, 3, or 4 were used to predict ADG between visits 1 and 5, and between visits 1 and 7. Logistic regression-based model accuracy results for piglet average daily gain (ADG) using piglet fecal microbiome data at different visits (i.e., 1, 2, 3, or 4), either singularly or in combinations are summarized in Table 8.

TABLE 8
Logistic regression-based model accuracy results
for piglet average daily gain (ADG)

| | | Test set accuracy |
|---|---|---|
| Model input | Model output | (%) |
| Piglet fecal microbiome at visits 1-3 | ADG between visit 1-5 | 60 |
| Piglet fecal microbiome at visits 1-3 | ADG between visit 1-7 | 74 |
| Piglet fecal microbiome at visit 1 | ADG between visit 1-5 | 53 |
| Piglet fecal microbiome at visit 1 | ADG between visit 1-7 | 59 |
| Piglet fecal microbiome at visit 2 | ADG between visit 1-5 | 56 |
| Piglet fecal microbiome at visit 2 | ADG between visit 1-7 | 65 |
| Piglet fecal microbiome at visit 3 | ADG between visit 1-5 | 60 |
| Piglet fecal microbiome at visit 3 | ADG between visit 1-7 | 78 |
| Piglet fecal microbiome at visit 4 | ADG between visit 1-5 | 64 |
| Piglet fecal microbiome at visit 4 | ADG between visit 1-7 | 78 |

The top 25 microbes most strongly associated with an increased or a decreased probability of high ADG using the visit 3 microbiome as input is summarized in Table 9.

TABLE 9
Top 25 Microbes of Visit 3 Microbiome Associated with
Increased and Decreased Probability of High ADG

| Rank | Increases probability of high ADG | Decreases probability of high ADG |
|---|---|---|
| 1 | SP3_e08 | Peptococcus |
| 2 | Romboutsia | [Eubacterium]_ruminantium_group |
| 3 | Asaccharospora | Actinobacillus |
| 4 | Sanguibacteroides | Hungatella |
| 5 | Oscillibacter | Monoglobus |
| 6 | Coriobacteriaceae_UCG_003 | [Clostridium]_innocuum_group |
| 7 | Coprococcus | Arcanobacterium |
| 8 | Blautia | Phascolarctobacterium |
| 9 | Subdoligranulum | Oribacterium |
| 10 | Catabacter | Parasutterella |
| 11 | Filifactor | Pygmaiobacter |
| 12 | Butyricicoccus | Turicibacter |
| 13 | Elusimicrobium | Succinivibrio |
| 14 | Allisonella | Holdemanella |
| 15 | Cerasicoccus | Quinella |
| 16 | Peptoniphilus | Acinetobacter |
| 17 | Anaerofilum | Akkermansia |
| 18 | Solobacterium | Synergistes |
| 19 | Cutibacterium | Lachnospiraceae_UCG_004 |
| 20 | Lachnospira | Sphaerochaeta |
| 21 | CAG_873 | Lachnospiraceae_UCG_002 |
| 22 | Eubacterium | Propionispira |
| 23 | Telluria | p 1088 a5 gut group |
| 24 | Lachnospiraceae_UCG_008 | [Eubacterium] nodatum group |
| 25 | Candidatus_{—}Soleaferrea | GCA_900066755 |

The top 25 microbes most strongly associated with an increased or a decreased probability of high ADG using the visit 4 microbiome as input is summarized in Table 10.

TABLE 10
Top 25 Microbes of Visit 4 Microbiome Associated with
Increased and Decreased Probability of High ADG

| Rank | Increases probability of high ADG | Decreases probability of high ADG |
|---|---|---|
| 1 | [Eubacterium]_eligens_group | Ruminococcus |
| 2 | Campylobacter | Bacteroides |
| 3 | Prevotella | Anaerosporobacter |
| 4 | [Eubacterium] fissicatena group | Prevotellaceae UCG 001 |
| 5 | Anaeroplasma | Bacillus |
| 6 | HT002 | Caproiciproducens |
| 7 | Akkermansia | Schwartzia |
| 8 | Ruminobacter | Alloprevotella |
| 9 | Lachnospira | V9D2013_group |
| 10 | Lachnospiraceae_NK4A136_group | Syntrophococcus |
| 11 | Parasutterella | Fusobacterium |
| 12 | Tyzzerella | Neisseria |
| 13 | Anaerobiospirillum | Succinivibrionaceae_UCG_002 |
| 14 | GCA_900066575 | Moraxella |
| 15 | Lachnospiraceae AC2044 group | Prevotellaceae UCG 004 |
| 16 | Acetitomaculum | Family_XIII_AD3011_group |
| 17 | CAG_56 | Catenibacterium |
| 18 | Oscillospira | Porphyromonas |
| 19 | Succiniclasticum | Conservatibacter |
| 20 | Intestinibacter | Lachnobacterium |
| 21 | Lachnospiraceae_UCG_007 | Erysipelotrichaceae_UCG_006 |
| 22 | Arcanobacterium | Sutterella |
| 23 | Prevotella_9 | [Eubacterium]_ruminantium_group |
| 24 | Oscillibacter | Alistipes |
| 25 | Alysiella | Lachnospiraceae UCG 008 |

The results indicate that highest test set accuracy was observed when predicting ADG from visits 1 to 7, regardless of which samples or sets of samples were used as model inputs (as tabulated in Table 8). Within the ADG from visits 1 to 7 models, the best performance was seen when using visits 3 or 4 as inputs (accuracy=78%), suggesting that the piglet microbiome around weaning can be used as a key indicator of future performance. In addition, logistic regression model coefficients were used to identify microbes that were strongly associated with high or low ADG, as tabulated in Table 9 and Table 10, respectively.

Sow models: by leveraging the vaginal microbiome samples and fecal microbiome samples, information was used to a.) determine which sow sample type (vaginal or fecal) would be best for predicting sow reproductive performance and piglet outcomes; and b.) assess whether the microbiome of the sow (vaginal and/or fecal) can be used to predict microbiome development in piglets.

The piglet microbiome was characterized using the Shannon diversity index at each sampling time point from each visit, noting that a more diverse microbial composition is generally associated with improved gut health. As classification models require a categorical target, the Shannon diversity index values were dichotomized into high and low diversity around the observed median. Since there were 3 to 4 piglets sampled per sow, there were multiple targets for each set of sow samples.

In addition to piglet gut microbiome development, we assessed whether the sow's microbiome could be used to predict reproductive performance at farrowing. Reproductive performance was measured using litter size and proportion of live births (i.e., live births/total litter size). These outcomes were also both dichotomized into high and low categories around the observed median.

Various models were developed, including 1.) fecal and vaginal microbiome samples: or vaginal microbiome samples only from sow to predict Shannon diversity in individual piglets of that sow; 2.) fecal and vaginal microbiome samples; or vaginal microbiome samples only from sow to predict litter size; and 3.) fecal and vaginal microbiome samples: or vaginal microbiome samples only from sow to predict proportion of live births.

Logistical regression-based model accuracy results for piglet Shannon alpha diversity measures using sow fecal and/or vaginal microbiome data are summarized in Table 11.

TABLE 11
Logistic Regression-Based Model Accuracy
for Piglet Shannon Alpha Diversity

| | | Test |
|---|---|---|
| Model | Model | set accuracy |
| input | output | (%) |
| Sow fecal + vaginal | Piglet Shannon alpha | 51 |
| microbiome | div. at visit 1 | |
| Sow fecal + vaginal | Piglet Shannon alpha | 57 |
| microbiome | div. at visit 2 | |
| Sow fecal + vaginal | Piglet Shannon alpha | 53 |
| microbiome | div. at visit 3 | |
| Sow fecal + vaginal | Piglet Shannon alpha | 55 |
| microbiome | div. at visit 4 | |
| Sow vaginal | Piglet Shannon alpha | 55 |
| microbiome | div. at visit 1 | |
| Sow vaginal | Piglet Shannon alpha | 54 |
| microbiome | div. at visit 2 | |
| Sow vaginal | Piglet Shannon alpha | 57 |
| microbiome | div. at visit 3 | |
| Sow vaginal | Piglet Shannon alpha | 56 |
| microbiome | div. at visit 4 | |

Predicting piglet microbiome in general revealed that test set accuracy was quite low for all models, however for the same output, accuracy was very similar or higher when using only the sow vaginal microbiome as input as shown in Table 11. These results indicate that the vaginal microbiome of the sow is likely the key indicator to measure when predicting the piglet gut microbiome.

Logistical regression-based model accuracy results for litter size and proportion of live births using sow fecal and/or vaginal microbiome data are summarized in Table 12.

TABLE 12
Logistic Regression-Based Model Accuracy for Piglet
Litter Size and Proportion of Live Births

| | | Test |
|---|---|---|
| Model | Model | set accuracy |
| input | output | (%) |
| Sow fecal + | Litter size | 61 |
| vaginal microbiome | | |
| Sow fecal + | Proportion live births | 52 |
| vaginal microbiome | | |
| Sow vaginal microbiome | Litter size | 61 |
| Sow vaginal microbiome | Proportion live births | 61 |

Predicting sow reproductive performance in general revealed that while test set accuracy was low for all models, the results are similar to the models for piglet microbiome diversity—using the sow vaginal microbiome results in similar or better results than using both sow vaginal and fecal microbiome samples as shown in Table 12.

The data show that there are metagenomic differences and performance metrics between piglets across different regions, farming type (conventional versus raised without antibiotics), before and after weaning, between different ages amongst others. There were associations between sow vaginal and/or fecal microbiome and piglet fecal microbiome. Microbes were identified that were associated with body weight gain in general and specific microbes associated with very high body weight gain including Intestinibacter, Coprococcus and Lachnospiraceae NK4A136 group. Regression models were identified to predict body weight of piglets (adjusted R²~0.91) using the microbiome and/or metadata using regression models (e.g. as function of farm+parity+Sex+Visit+Age+Grouping+All Genus). Predictive models for piglet performance (e.g., body weight) and sow reproductive health (e.g., litter size and proportion of live birth), were used to identify models: for predicting piglet performance (using body weight as surrogate) it was found that the best ADG predictions were using microbiome data from visit 3 and/or visit 4 as inputs—microbiome around weaning seems especially important for growth rate prediction; at the pre-weaning stage, it was found that the visit 3 piglet microbiome data showed 78% accuracy (using logistic regression) in predicting ADG; and at the post-weaning stage, it was found that the visit 4 piglet microbiome data showed 78% accuracy (using logistic regression) in predicting ADG. Regardless of which piglet timepoint (determined by the visit no.) was used as input, model performance was always better for ADG from visits 1-7 than ADG from visits 1-5. Inclusion of sow fecal microbiome data does not improve model performance over sow vaginal microbiome data alone, both for predicting piglet alpha diversity and sow reproductive performance. Overall, these findings show the use of the microbiome (individually and/or in its entirety) of individual animals to predict future health measures or future performance measures of animals.

### Example 7: Case Study on Solutions for Low Sow Reproductive Performance and Low Piglet Performance

The following example investigates the application of the multigenerational microbiome systems and methods herein as applied to multiple herds of sows exhibiting low reproductive performance and low piglet performance, including low piglet livability. Microbiome samples are collected from sows using fecal, vaginal, and milk samples form a subset of sows (e.g., day −7 of farrowing for fecal samples and vaginal samples and days 0-3 for colostrum or milk samples, where n=20). Microbiome samples are collected from piglets using fecal samples from a subset of piglets collected at pre-weaning (e.g., day 14, n=20). Samples are subjected to metagenomics analysis using shotgun metagenomics on fecal samples and 16S rRNA gene sequencing of milk and vaginal samples.

Model selection and predictions: One or more of the following models from the repository is selected: a piglet growth rate model, a piglet average daily gain model, a piglet alpha diversity model, a piglet mortality model, a piglet general health model, a piglet gastrointestinal pathogen risk model, a piglet respiratory pathogen risk model, a piglet anti-microbial growth promoter model, a piglet microbiome composition model, a piglet microbiome functional model, a piglet microbiome network interaction model, a piglet microbiome stability model, a piglet microbiome robustness model, a piglet microbiome resilience model, a sow microbiome composition model, a sow microbiome functional model, a sow microbiome network interaction model, a sow microbiome stability model, a sow microbiome robustness model, a sow microbiome resilience model, a sow reproductive performance model, and a sow to piglet performance model.

Predictions obtained by applying the selected models can provide prioritized recommendations and interventions for multiple herds, referred to herein as Herds 1-5.

For Herd 1, multiple predictions are obtained, indicating intermediate gut, reproductive, and mammary microbiome performance at the sow level that translates into non-optimal colonization of piglet gut, and as such can lead to intermediate piglet performance and/or livability with low pathogen risk. Prioritized recommendations include strategies for promotion of microbiome performance at difference body sites both at the sow and piglet level using one or more postbiotic interventions. For example, the one or more recommended interventions for Herd 1 can include any of the following interventions outlined in Table 13.

TABLE 13
Herd 1 Recommended Interventions

| | Recommended Interventions, Herd 1 |
|---|---|
| 1. | Cargill standard gestation, lactation, and grow-finish diets, and Cargill Nurture ™ |
| | nursery diet, PLUS |
| 2. | XPC ™ postbiotic @ 500 ppm during gestation (pregnancy) period (day −110 to day −5 |
| | relative to farrowing date) |
| 3. | XPC ™ postbiotic @ 500 ppm during lactation period (day −5 to day 21 relative to |
| | farrowing date) |
| 4. | DIA-V SWLQ ™ postbiotic @ 15 mL/sow/day (day −5 to day 3 relative to farrowing |
| | date) |
| 5. | DIA-V Nursery ™ postbiotic @ 1000 ppm during piglets Phase 1 of nursery program |
| | (day 0 to day 7 postweaning) |
| 6. | DIA-V Nursery ™ postbiotic @1000 ppm during piglets Phase 2 of nursery program |
| | (day 7 to day 21 postweaning) |
| 7. | XPC ™ postbiotic @ 1000 ppm during piglets Phase 3 of nursery program (day 21 to |
| | day 42 postweaning) |
| 8. | XPC ™ postbiotics @ 1000 ppm during grow-finish program (day 42 postweaning |
| | until end of production cycle; 115 to 120 days) |

For Herd 2, multiple predictions are obtained, indicating low gut, reproductive, and mammary microbiome performance at the sow level that translates into non-optimal colonization of piglet gut, and as such can lead to low piglet performance and/or livability, with low to medium pathogen risk. Prioritized recommendations include strategies for promotion of sow and piglet microbiome performance at difference body sites both using one or more postbiotic interventions. For example, the one or more recommended interventions for Herd 2 can include any of the following interventions outlined in Table 14.

TABLE 14
Herd 2 Recommended Interventions

| | Recommended Interventions, Herd 2 |
|---|---|
| 1. | Cargill standard gestation, lactation, and grow-finish diets, and Cargill Nurture ™ |
| | nursery diet, PLUS |
| 2. | XPC ™ postbiotic @ 500 ppm during gestation (pregnancy) period (day −110 to day −5 |
| | relative to farrowing date) |
| 3. | XPC ™ postbiotic @ 1000 ppm during lactation period (day −5 to day 21 relative to |
| | farrowing date) |
| 4. | DIA-V SWLQ ™ postbiotic @ 15 mL/sow/day (day −5 to day 7 relative to farrowing |
| | date) |
| 5. | DIA-V Nursery ™ postbiotic @ 1500 ppm during piglets Phase 1 of nursery program |
| | (day 0 to day 7 postweaning) |
| 6. | DIA-V Nursery ™ postbiotic @1000 ppm during piglets Phase 2 of nursery program |
| | (day 7 to day 21 postweaning) |
| 7. | XPC ™ postbiotic @ 1000 ppm during piglets Phase 3 of nursery program (day 21 to |
| | day 42 postweaning) |
| 8. | XPC ™ postbiotic @ 1000 ppm during grow-finish program (day 42 postweaning |
| | until end of production cycle; 115 to 120 days) |

For Herd 3, multiple predictions are obtained, indicating low gut, reproductive, and mammary microbiome performance at the sow level that translates into non-optimal colonization of piglet gut, and as such can lead to low piglet performance and/or livability, with a medium to high pathogen risk. Prioritized recommendations include strategies for promotion of sow and piglet microbiome performance at difference body sites both at the sow and piglet level using one or more postbiotic interventions, and for the suppression of potential gastrointestinal pathogens using one or more essential oils as interventions at the piglet level. For example, the one or more recommended interventions for Herd 3 can include any of the following interventions outlined in Table 15.

TABLE 15
Herd 3 Recommended Interventions

| | Recommended Interventions, Herd 3 |
|---|---|
| 1. | Cargill standard gestation, lactation, and grow-finish diets, and Cargill Nurture |
| | Boost ™ nursery diet, PLUS |
| 2. | XPC ™ postbiotic @ 500 ppm during gestation (pregnancy) period (day −110 to day −5 |
| | relative to farrowing date) |
| 3. | XPC ™ postbiotic @ 1500 ppm during lactation period (day −5 to day 21 relative to |
| | farrowing date) |
| 4. | DIA-V SWLQ ™ postbiotic @ 15 mL/sow/day (day −5 to day 7 relative to farrowing |
| | date) |
| 5. | DIA-V Nursery ™ postbiotic @ 1500 ppm during piglets Phase 1 of nursery program |
| | (day 0 to day 7 postweaning) |
| 6. | DIA-V Nursery ™ postbiotic @1500 ppm during piglets Phase 2 of nursery program |
| | (day 7 to day 21 postweaning) |
| 7. | PLUS |
| 8. | Cynergy ™ essential oil @ 400 ppm during Phase 1 and 2 nursery program (day 0 to |
| | day 21 postweaning) |
| 9. | XPC ™ postbiotic @ 1000 ppm during piglets Phase 3 of nursery program (day 21 to |
| | day 42 postweaning) |
| 10. | NaturSafe ™ postbiotic @ 1000 ppm during grow-finish program (day 42 |
| | postweaning until end of production cycle: 115 days to 120 days) |

For Herd 4, multiple predictions are obtained, indicating low gut, reproductive, and mammary microbiome performance at the sow level that translates into non-optimal colonization of piglet gut, and as such can lead to low piglet performance and/or livability, where pathogen risk is identified as high with greater susceptibility for Escherichia coli (gastrointestinal pathogen risk group) invasion. Prioritized recommendations include strategies for promotion of microbiome performance at difference body sites using one or more postbiotic interventions, and suppression of Escherichia coli at the piglet gut using one or more phytogenics or essential oils as interventions. For example, the one or more recommended interventions for Herd 4 can include any of the following interventions outlined in Table 16.

TABLE 16
Herd 4 Recommended Interventions

| | Recommended Interventions, Herd 4 |
|---|---|
| 1. | Cargill standard gestation, lactation, and grow-finish diets, and Cargill Nurture |
| | Boost ™ nursery diet, PLUS |
| 2. | XPC ™ postbiotic @ 750 ppm during gestation (pregnancy) period (day −110 to day −5 |
| | relative to farrowing date) |
| 3. | XPC ™ postbiotic @ 2000 ppm during lactation period (day −5 to day 21 relative to |
| | farrowing date) |
| 4. | DIA-V SWLQ ™ postbiotic @ 15 mL/sow/day (day −5 to day 7 relative to farrowing |
| | date) |
| 5. | DIA-V Nursery ™ postbiotic @ 2000 ppm during piglets Phase 1 of nursery program |
| | (day 0 to day 7 postweaning) |
| 6. | DIA-V Nursery ™ postbiotic @1500 ppm during piglets Phase 2 of nursery program |
| | (day 7 to day 21 postweaning) |
| 7. | PLUS |
| 8. | Option 1: Cynergy ™ essential oil @ 400 ppm during Phase 1 and 2 nursery program |
| | (day 0 to day 21 postweaning) |
| 9. | Option 2: Fresta Protect ™ phytogenic @ 1000 ppm during Phase 1 and 2 nursery |
| | program (day 0 to day 21 postweaning) |
| 10. | NaturSafe ™ postbiotic @ 1000 ppm during piglets Phase 3 of nursery program (day |
| | 21 to day 42 postweaning) |
| 11 | NaturSafe ™ postbiotic @ 1000 ppm during grow-finish program (day 42 |
| | postweaning until end of production cycle; 115 days to 120 days) |

For Herd 5, multiple predictions are obtained, indicating low gut, reproductive, and mammary microbiome performance at the sow level that translates into non-optimal colonization of piglet gut, and as such can lead to low piglet performance and/or livability, where pathogen risk is indicated as high with greater susceptibility for Streptococcus suis (gastrointestinal pathogen risk group) invasion. Prioritized recommendations include strategies for promotion of microbiome performance at difference body sites and suppression of Streptococcus suis at the piglet gut using one or more postbiotic interventions. For example, the one or more recommended interventions for Herd 5 can include any of the following interventions outlined in Table 17.

TABLE 17
Herd 5 Recommended Interventions

| | Recommended Interventions, Herd 5 |
|---|---|
| 1. | Cargill standard gestation, lactation, and grow-finish diets, and Cargill Nurture Low |
| | Complexity ™ nursery diet, PLUS |
| 2. | XPC ™ postbiotic @ 750 ppm during gestation (pregnancy) period (day −110 to day −5 |
| | relative to farrowing date) |
| 3. | XPC ™ postbiotic @ 2000 ppm during lactation period (day −5 to day 21 relative to |
| | farrowing date |
| 4. | DIA-V SWLQ ™ postbiotic @ 15 mL/sow/day (day −5 to day 7 relative to farrowing |
| | date) |
| 5. | DIA-V Nursery ™ @ 2000 ppm during piglets Phase 1 of nursery program (day 0 to |
| | day 7 postweaning) |
| 6. | DIA-V Nursery ™ @1500 ppm during piglets Phase 2 of nursery program (day 7 to |
| | day 21 postweaning) |
| 7. | PLUS |
| 8. | Option 1: Cynergy ™ essential oil @ 400 ppm during Phase 1 and 2 nursery program |
| | (day 0 to day 21 postweaning) |
| 9. | Option 2: Fresta Protect ™ phytogenic @ 1000 ppm during Phase 1 and 2 nursery |
| | program (day 0 to day 21 postweaning) |
| 10. | NaturSafe ™ postbiotic @ 1000 ppm during piglets Phase 3 of nursery program (day |
| | 21 to day 42 postweaning) |
| 11. | NaturSafe ™ postbiotic @ 1000 ppm during grow-finish program (day 42 |
| | postweaning until end of production cycle; 115 days to 120 days) |

The definitions described herein apply to all aspects as described unless otherwise stated.

In this document, the terms “a,” “an,” or “the” are used to include one or more than one unless the context clearly dictates otherwise. The term “or” is used to refer to a nonexclusive “or” unless otherwise indicated. All publications, patents, and patent documents referred to in this document are incorporated by reference herein in their entirety, as though individually incorporated by reference. In the event of inconsistent usages between this document and those documents so incorporated by reference, the usage in the incorporated reference is to be considered supplementary to that of this document: for irreconcilable inconsistencies, the usage in this document controls.

Values expressed in a range format are to be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” is to be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

Unless expressly stated, ppm (parts per million), percentage, and ratios are on a by weight basis. Percentage on a by weight basis (%, w/w or w/w %) is also referred to as weight percent (wt. %) or percent by weight (% wt.) herein.

### ADDITIONAL EXAMPLES

The following are non-limiting examples of the invention.

Example 1. A method for optimizing future health or future performance in an animal comprising: obtaining a sample dataset indicative of an entire microbial community within a gastrointestinal tract, mammary gland, reproductive system, or respiratory system of the animal; defining a query comprising one or more target queries for future health or future performance of the animal: selecting a model from a model repository based on the sample dataset and the query comprising the one or more targets; executing the selected model using the sample dataset and the query comprising the one or more targets to make predictions, generate recommendations, or generate interventions about future health or future performance of the animal; reporting the predictions, recommendations, or interventions about future health or future performance, or combinations thereof; and implementing one or more interventions as one or more adjustments to the animal's nutrition, management system, health care, or rearing environment based on the predictions, recommendations, or interventions.

Example 2. The method of example 1, wherein the sample data set comprises metagenomics data.

Example 3. The method of any one of examples 1 or 2, wherein the sample data set comprises metagenomics data and metadata.

Example 4. The method of any one of examples 1-3, wherein the sample data set comprises metagenomics data obtained using one or more sequencing techniques comprising shotgun DNA-based or RNA-based sequencing, 16S ribosomal RNA gene sequencing, 18S ribosomal RNA gene sequencing, or internal transcribed spacer amplicon sequencing.

Example 5. The method of any one of examples 1-4, wherein the sample dataset comprises metagenomics data sourced from at least one microbiome sample comprising a fecal sample, a rectal sample, a vaginal sample, a nasal sample, an oral sample, a lung sample, or a mammary gland sample.

Example 6. The method of any one of examples 1-5, wherein the sample dataset comprises metadata for the animal comprising one or more of body weight, birth weight, animal breed, animal sex, body composition, growth rate, feed conversion ratio, mortality, morbidity, livability, illness history, health and performance measures, reproductive measures, current or prior disease states, gastrointestinal pathogen risk, respiratory pathogen risk, reproductive system pathogen risk, mammary glands pathogen risk, feed type, vaccinations administered and date of vaccination administration, supplement type, use of anti-microbial resistance promoters, conventional rearing, rearing animals raised with antimicrobials, geographical location, rearing conditions, animal life stage, microbiome sample type, microbiome sampling life stage, microbiome sampling age or time, metagenomics method used, farm location, herd size, animal heart girth measurement, or nutrition type.

Example 7. The method of any one of examples 1-6, wherein the targets queries for future health or future performance comprise one or more of piglet growth rate, piglet average daily weight gain, piglet microbiome alpha diversity, piglet livability, piglet mortality, piglet general health, piglet gastrointestinal pathogen risk, piglet respiratory pathogen risk, piglet anti-microbial growth promoter use, piglet microbiome composition, piglet microbiome function, piglet microbiome network interaction, piglet microbiome stability, piglet microbiome robustness, and/or piglet microbiome resilience: or one or more of sow livability, sow mortality, sow general health, sow gastrointestinal pathogen risk, sow respiratory pathogen risk, sow reproductive system pathogen risk, sow mammary glands pathogen risk, sow anti-microbial growth promoter use, sow microbiome composition, sow microbiome function, sow microbiome network interaction, sow microbiome stability, sow microbiome robustness, sow microbiome resilience, sow reproductive performance, and/or sow to piglet ratios; or one or more of gilt livability, gilt mortality, gilt general health, gilt gastrointestinal pathogen risk, gilt respiratory pathogen risk, gilt reproductive system pathogen risk, gilt anti-microbial growth promoter use, gilt microbiome composition, gilt microbiome function, gilt microbiome network interaction, gilt microbiome stability, gilt microbiome robustness, gilt microbiome resilience, and/or gilt future reproductive performance.

Example 8. The method of any one of examples 1-7, wherein the model selected from the model repository comprises one or more trained models comprising a prediction model, a recommendation model, or an intervention model.

Example 9. The method of any one of examples 1-8, wherein the model selected from the model repository comprises a prediction model.

Example 10. The method of example 9, wherein the prediction model comprises one or more predictions regarding the future performance or the future health of the animal; and wherein the one or more predictions comprise a prediction of future body weight, future feed conversion ratio, future feed intake, future growth rate, future average daily weight gain, future heart girth diameter, future livability, future mortality, future morbidity, future gastrointestinal pathogen risk, future respiratory pathogen risk, future reproductive system pathogen risk, future mammary glands pathogen risk, future litter size, future number of liveborn piglets, future number of stillborn piglets, future parity, future sow mortality, or future incidence of uterine prolapse.

Example 11. The method of any one of examples 1-8, wherein the model selected from the model repository comprises a recommendation model.

Example 12. The method of example 11, wherein the recommendation model comprises one or more recommendations regarding the future performance or the future health of the animal; and wherein the one or more recommendations comprises a recommendation to: change a feed or supplement composition, add or remove a feed or supplement composition, administer one or more vaccines, administer one or more medications, make alterations to a rearing environment or management system, select an animal comprising an indication for enhanced future performance or future health, select a gilt comprising an indication for enhanced future performance or future health, or add or remove anti-microbial growth promoters into a diet of the animal.

Example 13. The method of any one of examples 1-8, wherein the model selected from the model repository comprises an intervention model.

Example 14. The method of example 13, wherein the intervention model comprises one or more interventions regarding the future performance or the future health of the animal; and wherein the one or more interventions comprise: changing a feed or supplement composition, supplying a feed or supplement composition, adding or removing a feed or supplement composition, administering one or more vaccines, administering one or more medications, making alterations to the rearing environment and management system, making alterations to the management system, selecting an animal comprising an indication for enhanced future performance or future health, selecting a gilt comprising an indication for enhanced future performance or future health, or adding or removing one or more anti-microbial growth promoters into a diet of the animal.

Example 15. The method of any one of examples 1-14, wherein the predictions, recommendations, or interventions are configured to reduce the incidence or severity of disease, improve health and performance measures in the animal, reduce the number of animals needing to be culled from a population, select individual animals comprising an indication for enhanced future performance or future health, identify individual animals that require interventions, or reduce the reliance on antimicrobial medications.

Example 16. The method of any one of examples 1-15, wherein the model has been generated based on metagenomics data or metadata from one or more past generations of animals.

Example 17. The method of any one of examples 1-16, wherein the model further has been generated based on the type of metagenomics data and the body site where the sampled data was obtained.

Example 18. The method of any one of examples 1-17, wherein the model comprises one or more of a piglet model, a sow model, or a gilt model.

Example 19. The method of example 18, further comprising a piglet model, wherein the piglet model comprises one or more of: a piglet growth rate model, a piglet average daily gain model, a piglet alpha diversity model, a piglet mortality model, a piglet livability model, a piglet general health model, a piglet gastrointestinal pathogen risk model, a piglet respiratory pathogen risk model, a piglet anti-microbial growth promoter model, a piglet biotic health and growth promoter model, a piglet microbiome composition model, a piglet microbiome functional model, a piglet microbiome network interaction model, a piglet microbiome stability model, a piglet microbiome robustness model, and a piglet microbiome resilience model.

Example 20. The method of example 18, further comprising a sow model, wherein the sow model comprises one or more of: a sow mortality model, a sow livability model, a sow general health model, a sow gastrointestinal pathogen risk model, a sow respiratory pathogen risk model, a sow reproductive system risk model, a sow mammary glands pathogen risk model, a sow anti-microbial growth promoter risk model, a sow biotic health and growth promoter model, a sow microbiome composition model, a sow microbiome functional model, a sow microbiome network interaction model, a sow microbiome stability model, a sow microbiome robustness model, a sow microbiome resilience model, a sow reproductive performance model, and a sow to piglet performance model.

Example 21. The method of example 18, further comprising a gilt model, wherein the gilt model comprises one or more of: a gilt mortality model, a gilt livability model, a gilt general health model, a gilt gastrointestinal pathogen risk model, a gilt respiratory pathogen risk model, a gilt reproductive system risk model, a gilt anti-microbial growth promoter risk model, a gilt biotic health and growth promoter model, a gilt microbiome composition model, a gilt microbiome functional model, a gilt microbiome network interaction model, a gilt microbiome stability model, a gilt microbiome robustness model, a gilt microbiome resilience model, and a gilt future reproductive performance model.

Example 22. The method of any one of examples 1-21, wherein the animal comprises a swine animal.

Example 23. The method of any one of examples 1-22, wherein the animal comprises a piglet, a gilt, or a sow.

Example 24. The method of any one of examples 1-23, wherein obtaining a sample dataset further comprises obtaining a sample dataset from more than one animal.

Example 25. The method of any one of examples 1-24, wherein implementing the one or more interventions as one or more adjustments comprises: switching from conventional rearing to an environment where the one or more animals are raised without antimicrobials; switching from an environment where the one or more animals are raised without antimicrobials to a conventional rearing with antimicrobials; switching to a system of rearing where the one or more animals are raised using alternatives to antimicrobial compounds comprising prebiotics, probiotics, postbiotics, or phytogenics; increasing or decreasing herd size; implementing one or more culling decisions; isolating one or more animals; adding or removing one or more supplement compositions; changing an existing supplement composition; adding or removing a supplement containing one or more anti-microbial growth promoters; adding or removing a feed composition or compositions to address one or more nutrition deficiencies; changing a feed composition; adding of one or more vitamins or minerals; adding or removing one or more anti-microbial growth promoters into a feed; administering one or more vaccines to prevent or treat a disease; or administering one or more medications to prevent or treat a disease.

Example 26. A multigenerational microbiome system for optimizing future health or future performance in one or more animals comprising: a metagenomics component configured to receive metagenomics data obtained from a microbiome sample of the one or more animals; a metadata acquisition component configured to receive metadata about the one or more animals; a query acquisition component configured to receive one or more target queries for future health or future performance of the one or more animals; a processing component comprising a model selection engine, the model selection engine configured to select a model or set of models from a model repository based on the metagenomics data, the metadata, and the target query: a prediction generation engine configured to execute the selected model or set of models to generate one or more predictions about the future health or future performance of the animal; a recommendation prioritization engine configured to use the predictions to identify an animal or animals at risk for future adverse health or future adverse performance, and to generate one or more recommendations that are prioritized to address the identified risk; and an intervention prioritization engine configured to use the one or more predictions or recommendations to generate one or more interventions that are prioritized to address the identified risk; a report generation engine configured to receive the predictions, recommendations, or interventions and generate one or more reports that include the specific predictions, recommendations, or interventions, or a combination thereof, and a detailed rationale about adjustments suitable for optimizing the future health or future performance in an animal or group of animals; and an adjustment component configured to implement the one or more interventions as one or more adjustments to the animal or group of animals.

Example 27. The system of example 26, further comprising a model repository configured to store one or more trained models, target queries, profiles, model performance metrics, or parameters.

Example 28. The system of any one of examples 26 or 27, further comprising a data repository configured to store the metagenomics data or the metadata.

Example 29. The system of any one of examples 26-28, wherein implementing the one or more interventions as one or more adjustments to the animal or group of animals comprise: switching from conventional rearing to an environment where the one or more animals are raised without antimicrobials: switching from an environment where the one or more animals are raised without antimicrobials to a conventional rearing with antimicrobials; switching to a system of rearing where the one or more animals are raised using alternatives to antimicrobial compounds comprising prebiotics, probiotics, postbiotics, or phytogenics; increasing or decreasing herd size; implementing one or more culling decisions; isolating one or more animals; adding or removing one or more supplement compositions; changing an existing supplement composition; adding or removing a supplement containing one or more anti-microbial growth promoters; adding or removing a feed composition or compositions to address one or more nutrition deficiencies; changing a feed composition; adding of one or more vitamins or minerals; adding or removing one or more anti-microbial growth promoters into a feed; administering one or more vaccines to prevent or treat a disease; or administering one or more medications to prevent or treat a disease.

Example 30. The system of any of examples 26-29, wherein the metagenomics data comprises one or more of shotgun DNA-based or RNA-based sequencing data, 16S ribosomal RNA gene sequencing data, 18S ribosomal RNA gene sequencing data, or internal transcribed spacer amplicon sequencing data.

Example 31. The system of any of examples 26-30, wherein the metadata comprises one or more of body weight, birth weight, animal breed, animal sex, body composition, growth rate, feed conversion ratio, mortality, morbidity, livability, illness history, health and performance measures, reproductive measures, current or prior disease states, gastrointestinal pathogen risk, respiratory pathogen risk, reproductive system pathogen risk, mammary glands pathogen risk, feed type, vaccinations administered and date of vaccination administration, supplement type, use of anti-microbial resistance promoters, conventional rearing, rearing animals raised with antimicrobials, geographical location, rearing conditions, animal life stage, microbiome sample type, microbiome sampling life stage, microbiome sampling age or time, metagenomics method used, farm location, herd size, animal heart girth measurement, or nutrition type.

Example 32. The system of any of examples 26-31, wherein the targets queries for future health or future performance comprise: one or more of piglet growth rate, piglet average daily weight gain, piglet microbiome alpha diversity, piglet livability, piglet mortality, piglet general health, piglet gastrointestinal pathogen risk, piglet respiratory pathogen risk, piglet anti-microbial growth promoter use, piglet microbiome composition, piglet microbiome function, piglet microbiome network interaction, piglet microbiome stability, piglet microbiome robustness, and/or piglet microbiome resilience; or one or more of sow livability, sow mortality, sow general health, sow gastrointestinal pathogen risk, sow respiratory pathogen risk, sow reproductive system pathogen risk, sow mammary glands pathogen risk, sow anti-microbial growth promoter use, sow microbiome composition, sow microbiome function, sow microbiome network interaction, sow microbiome stability, sow microbiome robustness, sow microbiome resilience, sow reproductive performance, and/or sow to piglet ratios; or one or more of gilt livability, gilt mortality, gilt general health, gilt gastrointestinal pathogen risk, gilt respiratory pathogen risk, gilt reproductive system pathogen risk, gilt anti-microbial growth promoter use, gilt microbiome composition, gilt microbiome function, gilt microbiome network interaction, gilt microbiome stability, gilt microbiome robustness, gilt microbiome resilience, and/or gilt future reproductive performance.

Example 33. The system of any of examples 26-32, wherein the model selected from the model repository comprises one or more trained models comprising a prediction model, a recommendation model, or an intervention model.

Example 34. The system of any of examples 26-33, wherein the model selected from the model repository comprises a prediction model.

Example 35. The system of example 34, wherein the prediction model comprises one or more predictions regarding the future performance or the future health of the animal; and wherein the one or more predictions comprise a prediction of future body weight, future feed conversion ratio, future feed intake, future growth rate, future average daily weight gain, future heart girth diameter, future livability, future mortality, future morbidity, future gastrointestinal pathogen risk, future respiratory pathogen risk, future reproductive system pathogen risk, future mammary glands pathogen risk, future litter size, future number of liveborn piglets, future number of stillborn piglets, future parity, future sow mortality, or future incidence of uterine prolapse.

Example 36. The system of any of examples 26-33, wherein the model identified from the model repository comprises a recommendation model.

Example 37. The system of example 36, wherein the recommendation model comprises one or more recommendations regarding the future performance or the future health of the animal; and wherein the one or more recommendations comprises a recommendation to: change a feed or supplement composition, add or remove a feed or supplement composition, administer one or more vaccines, administer one or more medications, make alterations to a rearing environment or management system, select an animal comprising an indication for enhanced future performance or future health, select a gilt comprising an indication for enhanced future performance or future health, or add or remove anti-microbial growth promoters into a diet of the animal.

Example 38. The system of any of examples 26-33, wherein the model identified from the model repository comprises an intervention model.

Example 39. The method of example 38, wherein the intervention model comprises one or more interventions regarding the future performance or the future health of the animal; and wherein the one or more interventions comprise: changing a feed or supplement composition, supplying a feed or supplement composition, adding or removing a feed or supplement composition, administering one or more vaccines, administering one or more medications, making alterations to the rearing environment and management system, making alterations to the management system, selecting an animal comprising an indication for enhanced future performance or future health, selecting a gilt comprising an indication for enhanced future performance or future health, or adding or removing one or more anti-microbial growth promoters into a diet of the animal.

Example 40. The system of any of examples 26-39, wherein the predictions, recommendations, or interventions are configured to reduce the incidence or severity of disease, improve health and performance measures in the animal, reduce the number of animals needing to be culled from a population, select individual animals comprising an indication for enhanced future performance or future health, identify individual animals that require interventions, or reduce the reliance on antimicrobial medications.

Example 41. The system of any of examples 26-40, wherein the model has been generated based on metagenomics data or metadata from one or more past generations of animals.

Example 42. The system of any of examples 26-41, wherein the model further has been generated based on the type of metagenomics data and the body site where the sampled data was obtained.

Example 43. The system of any of examples 26-42, wherein the model comprises one or more of a piglet model, a sow model, or a gilt model.

Example 44. The system of example 43, further comprising a piglet model, wherein the piglet model comprises one or more of: a piglet growth rate model, a piglet average daily gain model, a piglet alpha diversity model, a piglet mortality model, a piglet livability model, a piglet general health model, a piglet gastrointestinal pathogen risk model, a piglet respiratory pathogen risk model, a piglet anti-microbial growth promoter model, a piglet biotic health and growth promoter model, a piglet microbiome composition model, a piglet microbiome functional model, a piglet microbiome network interaction model, a piglet microbiome stability model, a piglet microbiome robustness model, and a piglet microbiome resilience model.

Example 45. The system of example 43, further comprising a sow model, wherein the sow model comprises one or more of: a sow mortality model, a sow livability model, a sow general health model, a sow gastrointestinal pathogen risk model, a sow respiratory pathogen risk model, a sow reproductive system risk model, a sow mammary glands pathogen risk model, a sow anti-microbial growth promoter risk model, a sow biotic health and growth promoter model, a sow microbiome composition model, a sow microbiome functional model, a sow microbiome network interaction model, a sow microbiome stability model, a sow microbiome robustness model, a sow microbiome resilience model, a sow reproductive performance model, and a sow to piglet performance model.

Example 46. The system of example 43, further comprising a gilt model, wherein the gilt model comprises one or more of: a gilt mortality model, a gilt livability model, a gilt general health model, a gilt gastrointestinal pathogen risk model, a gilt respiratory pathogen risk model, a gilt reproductive system risk model, a gilt anti-microbial growth promoter risk model, a gilt biotic health and growth promoter model, a gilt microbiome composition model, a gilt microbiome functional model, a gilt microbiome network interaction model, a gilt microbiome stability model, a gilt microbiome robustness model, a gilt microbiome resilience model, and a gilt future reproductive performance model.

Example 47. The system of any of examples 26-46, wherein the animal comprises a swine animal.

Example 48. The system of any of examples 26-47, wherein the animal comprises a piglet, a gilt, or a sow.

Example 49. The system of any of examples 26-48, wherein obtaining a sample dataset further comprises obtaining a sample dataset from more than one animal.

Example 50. A method for predicting future health or future performance in an animal comprising: obtaining a sample dataset indicative of an entire microbial community within a gastrointestinal tract, mammary gland, or reproductive system, or respiratory system of the animal; defining a query comprising one or more target queries for future health or future performance of the animal: selecting a model from a model repository based on the sample dataset and the query comprising the one or more targets; executing the selected model using the sample dataset and the query comprising the one or more targets to make predictions, generate recommendations, or generate interventions about future health or future performance of the animal; reporting the predictions, recommendations, or interventions about future health or future performance, or combinations thereof; and implementing one or more interventions as one or more adjustments to the animal's nutrition, management system, health care, or rearing environment based on the predictions, recommendations, or interventions; wherein each model selected from the model repository is generated by a model generation engine, and wherein the model generation engine has been trained using one or more of metagenomics data or metadata obtained from one or more observational and interventional studies.

Example 51. The method of example 50, wherein each model selected from the model repository is periodically retrained using newly acquired data obtained from one or more metagenomics data or metadata obtained from one or more observational and interventional studies, or from one or more metagenomics data or metadata obtained from one or more production farm locations.

Example 52. The method of any of examples 50 or 51, wherein implementing the one or more interventions as one or more adjustments comprises: switching from conventional rearing to an environment where the one or more animals are raised without antimicrobials; switching from an environment where the one or more animals are raised without antimicrobials to a conventional rearing with antimicrobials; switching to a system of rearing where the one or more animals are raised using alternatives to antimicrobial compounds comprising prebiotics, probiotics, postbiotics, or phytogenics; increasing or decreasing herd size; implementing one or more culling decisions; isolating one or more animals; adding or removing one or more supplement compositions; changing an existing supplement composition; adding or removing a supplement containing one or more anti-microbial growth promoters; adding or removing a feed composition or compositions to address one or more nutrition deficiencies; changing a feed composition; adding of one or more vitamins or minerals; adding or removing one or more anti-microbial growth promoters into a feed; administering one or more vaccines to prevent or treat a disease: or administering one or more medications to prevent or treat a disease.

Example 53. A method for optimizing future health or future performance in an animal comprising: obtaining a sample dataset indicative of an entire microbial community within a reproductive system of the animal; defining a query comprising one or more target queries for future health or future performance of the animal; selecting a model from a model repository based on the sample dataset and the query comprising the one or more targets: executing the selected model using the sample dataset and the query comprising the one or more targets to make predictions, generate recommendations, or generate interventions about future health or future performance of the animal; reporting the predictions, recommendations, or interventions about future health or future performance, or combinations thereof; and implementing one or more interventions as one or more adjustments to the animal's nutrition, management system, health care, or rearing environment based on the predictions, recommendations, or interventions.

Example 54. A method for optimizing future health or future performance in an animal comprising: obtaining a sample dataset indicative of an entire microbial community within a mammary gland of the animal: defining a query comprising one or more target queries for future health or future performance of the animal; selecting a model from a model repository based on the sample dataset and the query comprising the one or more targets; executing the selected model using the sample dataset and the query comprising the one or more targets to make predictions, generate recommendations, or generate interventions about future health or future performance of the animal; reporting the predictions, recommendations, or interventions about future health or future performance, or combinations thereof; and implementing one or more interventions as one or more adjustments to the animal's nutrition, management system, health care, or rearing environment based on the predictions, recommendations, or interventions.

Example 55. A method for optimizing future health or future performance in an animal comprising: obtaining a sample dataset indicative of an entire microbial community within a respiratory system of the animal; defining a query comprising one or more target queries for future health or future performance of the animal; selecting a model from a model repository based on the sample dataset and the query comprising the one or more targets; executing the selected model using the sample dataset and the query comprising the one or more targets to make predictions, generate recommendations, or generate interventions about future health or future performance of the animal: reporting the predictions, recommendations, or interventions about future health or future performance, or combinations thereof; and implementing one or more interventions as one or more adjustments to the animal's nutrition, management system, health care, or rearing environment based on the predictions, recommendations, or interventions.

1. A method for optimizing future health or future performance in an animal comprising:
obtaining a sample dataset indicative of an entire microbial community within a gastrointestinal tract, mammary gland, reproductive system, or respiratory system of the animal;
defining a query comprising one or more target queries for future health or future performance of the animal;
selecting a model from a model repository based on the sample dataset and the query comprising the one or more targets;
executing the selected model using the sample dataset and the query comprising the one or more targets to make predictions, generate recommendations, or generate interventions about future health or future performance of the animal;
reporting the predictions, recommendations, or interventions about future health or future performance, or combinations thereof; and
implementing one or more interventions as one or more adjustments to the animal's nutrition, management system, health care, or rearing environment based on the predictions, recommendations, or interventions.

2. The method of claim 1, wherein the sample data set comprises metagenomics data.

3. The method of claim 1, wherein the sample data set comprises metagenomics data and metadata.

4. The method of claim 1, wherein the sample data set comprises metagenomics data obtained using one or more sequencing techniques comprising shotgun DNA-based or RNA-based sequencing, 16S ribosomal RNA gene sequencing, 18S ribosomal RNA gene sequencing, or internal transcribed spacer amplicon sequencing.

5. The method of claim 1, wherein the sample dataset comprises metagenomics data sourced from at least one microbiome sample comprising a fecal sample, a rectal sample, a vaginal sample, a nasal sample, an oral sample, a lung sample, or a mammary gland sample.

6. The method of claim 1, wherein the sample dataset comprises metadata for the animal comprising one or more of body weight, birth weight, animal breed, animal sex, body composition, growth rate, feed conversion ratio, mortality, morbidity, livability, illness history, health and performance measures, reproductive measures, current or prior disease states, gastrointestinal pathogen risk, respiratory pathogen risk, reproductive system pathogen risk, mammary glands pathogen risk, feed type, vaccinations administered and date of vaccination administration, supplement type, use of anti-microbial resistance promoters, conventional rearing, rearing animals raised with antimicrobials, geographical location, rearing conditions, animal life stage, microbiome sample type, microbiome sampling life stage, microbiome sampling age or time, metagenomics method used, farm location, herd size, animal heart girth measurement, or nutrition type.

7. The method of claim 1, wherein the targets queries for future health or future performance comprise one or more of piglet growth rate, piglet average daily weight gain, piglet microbiome alpha diversity, piglet livability, piglet mortality, piglet general health, piglet gastrointestinal pathogen risk, piglet respiratory pathogen risk, piglet anti-microbial growth promoter use, piglet microbiome composition, piglet microbiome function, piglet microbiome network interaction, piglet microbiome stability, piglet microbiome robustness, and/or piglet microbiome resilience; or
one or more of sow livability, sow mortality, sow general health, sow gastrointestinal pathogen risk, sow respiratory pathogen risk, sow reproductive system pathogen risk, sow mammary glands pathogen risk, sow anti-microbial growth promoter use, sow microbiome composition, sow microbiome function, sow microbiome network interaction, sow microbiome stability, sow microbiome robustness, sow microbiome resilience, sow reproductive performance, and/or sow to piglet ratios; or
one or more of gilt livability, gilt mortality, gilt general health, gilt gastrointestinal pathogen risk, gilt respiratory pathogen risk, gilt reproductive system pathogen risk, gilt anti-microbial growth promoter use, gilt microbiome composition, gilt microbiome function, gilt microbiome network interaction, gilt microbiome stability, gilt microbiome robustness, gilt microbiome resilience, and/or gilt future reproductive performance.

8. The method of claim 1, wherein the model selected from the model repository comprises one or more trained models comprising a prediction model, a recommendation model, or an intervention model.

9. The method of claim 1, wherein the model selected from the model repository comprises a prediction model.

10. The method of claim 9, wherein the prediction model comprises one or more predictions regarding the future performance or the future health of the animal; and
wherein the one or more predictions comprise a prediction of: future body weight, future feed conversion ratio, future feed intake, future growth rate, future average daily weight gain, future heart girth diameter, future livability, future mortality, future morbidity, future gastrointestinal pathogen risk, future respiratory pathogen risk, future reproductive system pathogen risk, future mammary glands pathogen risk, future litter size, future number of liveborn piglets, future number of stillborn piglets, future parity, future sow mortality, or future incidence of uterine prolapse.

11. The method of claim 1, wherein the model selected from the model repository comprises a recommendation model.

12. The method of claim 11, wherein the recommendation model comprises one or more recommendations regarding the future performance or the future health of the animal; and
wherein the one or more recommendations comprises a recommendation to: change a feed or supplement composition, add or remove a feed or supplement composition, administer one or more vaccines, administer one or more medications, make alterations to a rearing environment or management system, select an animal comprising an indication for enhanced future performance or future health, select a gilt comprising an indication for enhanced future performance or future health, or add or remove anti-microbial growth promoters into a diet of the animal.

13. The method of claim 1, wherein the model selected from the model repository comprises an intervention model.

14. The method of claim 13, wherein the intervention model comprises one or more interventions regarding the future performance or the future health of the animal; and
wherein the one or more interventions comprise: changing a feed or supplement composition, supplying a feed or supplement composition, adding or removing a feed or supplement composition, administering one or more vaccines, administering one or more medications, making alterations to the rearing environment and management system, making alterations to the management system, selecting an animal comprising an indication for enhanced future performance or future health, selecting a gilt comprising an indication for enhanced future performance or future health, or adding or removing one or more anti-microbial growth promoters into a diet of the animal.

15. The method of claim 1, wherein the predictions, recommendations, or interventions are configured to reduce the incidence or severity of disease, improve health and performance measures in the animal, reduce the number of animals needing to be culled from a population, select individual animals comprising an indication for enhanced future performance or future health, identify individual animals that require interventions, or reduce the reliance on antimicrobial medications.

16. The method of claim 1, wherein the model further has been generated based on the type of metagenomics data and the body site where the sampled data was obtained.

17. The method of claim 1, wherein the model comprises one or more of a piglet model, a sow model, or a gilt model.

18. The method of claim 18, further comprising a piglet model, wherein the piglet model comprises one or more of: a piglet growth rate model, a piglet average daily gain model, a piglet alpha diversity model, a piglet mortality model, a piglet livability model, a piglet general health model, a piglet gastrointestinal pathogen risk model, a piglet respiratory pathogen risk model, a piglet anti-microbial growth promoter model, a piglet biotic health and growth promoter model, a piglet microbiome composition model, a piglet microbiome functional model, a piglet microbiome network interaction model, a piglet microbiome stability model, a piglet microbiome robustness model, and a piglet microbiome resilience model.

19. The method of claim 18, further comprising a sow model, wherein the sow model comprises one or more of: a sow mortality model, a sow livability model, a sow general health model, a sow gastrointestinal pathogen risk model, a sow respiratory pathogen risk model, a sow reproductive system risk model, a sow mammary glands pathogen risk model, a sow anti-microbial growth promoter risk model, a sow biotic health and growth promoter model, a sow microbiome composition model, a sow microbiome functional model, a sow microbiome network interaction model, a sow microbiome stability model, a sow microbiome robustness model, a sow microbiome resilience model, a sow reproductive performance model, and a sow to piglet performance model.

20. The method of claim 18, further comprising a gilt model, wherein the gilt model comprises one or more of: a gilt mortality model, a gilt livability model, a gilt general health model, a gilt gastrointestinal pathogen risk model, a gilt respiratory pathogen risk model, a gilt reproductive system risk model, a gilt anti-microbial growth promoter risk model, a gilt biotic health and growth promoter model, a gilt microbiome composition model, a gilt microbiome functional model, a gilt microbiome network interaction model, a gilt microbiome stability model, a gilt microbiome robustness model, a gilt microbiome resilience model, and a gilt future reproductive performance model.
